(19) United States
(12) Patent Application Publication
(10) Pub. No.: US 2005/0273017 A1
(43) Pub. Date: Dec. 8, 2005

(54) Collective brain measurement system and method

(76) Inventor: Evian Gordon, Vaucluse (AU)

Correspondence Address: ST. ONGE STEWARD JOHNSTON & REENS, LLC, 986 BEDFORD STREET, STAMFORD, CT, 06905-5619, US

(21) Appl. No.: 11/091,048

(22) Filed: Mar. 28, 2005

(30) Foreign Application Priority Data
Mar. 26, 2004 (AU) 2004901663

(51) Int. Cl. A61B 5/04

(52) U.S. Cl. 600/544

(57) ABSTRACT

A method of providing diagnosis capability, diagnosis of the effects of treatment or diagnosis of distinctive capabilities of a test subject, the method can comprise the steps of: (a) carrying out a series of tests on a group of subjects of at least two modal measures, the modal measures comprising brain-body function, brain structure, neuropsychological, personality, genetics, personal history, performance and behaviour; and (b) examining the inter-relationships between the modal measures to output an analysis of the inter-relationships of two or more measures of the tests results of the group of subjects.

This application claims priority of Australian Patent Application No. 2004901663 filed Mar. 26, 2004.

### FIELD OF THE INVENTION

The present invention relates to the field of performing brain measurements and, in particular, discloses a global system for brain analysis and functional disorder identification.

The invention has been developed primarily for use as a method of obtaining and collating data to be used as a comparative tool on a global scale for brain-related disease and dysfunction and will be described hereinafter with reference to this application. However it will be appreciated that the invention is not limited to this particular field of use.

### BACKGROUND OF THE INVENTION

Any discussion of the prior art throughout the specification should in no way be considered as an admission that such prior art is widely known or forms part of the common general knowledge in the field.

In the field of neuroscience, taking measurements of the brain and brain wave patterns can often lead to insights in the treatment of disease. Unfortunately, there is no structural standard by which measurements can be correlated and the result is that researchers are hampered by the lack of informative data with which to work.

Most research uses small subject numbers, a limited number of measures and methods of analysis. This makes it difficult to gauge the generality of the research. In particular, the interactions and inter-relationships between basic variables, such as gender, age and personality variables, cannot be controlled for.

Whilst there are numerous studies showing possible distinctive patterns of brain function in previous research, they have been undertaken using selective aspects of brain function, performance or behavior and usually in small databases (sample sizes of less than 20 in the case of brain function). It may be myopic to continue to generate large numbers of such outcomes, without some evaluation of the relative amount of variance explained by the factors such as age, gender and personality variables, since statistical control over these variables cannot be obtained in studies with sample sizes of less than 20.

Brain databases for medical purposes are rapidly being developed, but significant issues such as quality control and consistency of activation paradigms across laboratories limit progress for the clinical application of databases.

### SUMMARY OF THE INVENTION

It is an object of the invention in its preferred form to provide a method of obtaining and collating data to be used as a comparative tool on a global scale for brain-related disease and dysfunction, treatment assessment and/or determination of distinctive cognitive capabilities in a subject.

In accordance with a first aspect of the present invention, there is provided a method of providing diagnosis capability, diagnosis of the effects of treatment or diagnosis of distinctive capabilities of a test subject, the method can comprise the steps of: (a) carrying out a series of tests on a group of subjects of at least two modal measures, the modal measures comprising brain-body function, brain structure, neuropsychological, personality, genetics, personal history, performance and behavior; and (b) examining the inter-relationships between the modal measures to output an analysis of the inter-relationships of two or more measures of the tests results of the group of subjects.

Preferred embodiments also include the steps of: (c) examining a test subject on at least two of the modal measures; (d) analyzing the results of step (c) relative to the test results of the group of subjects to determine a distinctive pattern of results for the test subject. The test subject can be examined on the same series of tests as carried out on the group of subjects or alternatively the test subject can be examined on a subset of the series of tests as carried out on the group of subjects. The test subjects are preferably geographically dispersed. Preferably, the number of control subjects can be at least 100.

The method can also include the step of measuring of electromagnetic signals emanating from the subject's brain in response to various interactive tasks carried out by the test subject. The external stimuli can include a series of interactive tests conducted by the subject. The measured electromagnetic signals are preferably subjected to signal processing to extract measurements of at least one of delta, theta, alpha, beta gamma frequency ranges for comparison with corresponding ranges of the test subjects. The signals are normally also subject to detecting abnormal power levels in the frequency ranges and extracting event related potentials from the electromagnetic signals.

The interactive tests can include at least one of: a resting EEG test; a habituation paradigm test, an efficiency of target processing test, a visual tracking task, an inhibition test, a conscious and subconscious processing of facial emotions test, a memory and sustained attention test, a planning and error correction test an a fight and flight reflex test. The method can also include the step of conducting a gamma phase synchrony analysis of the electromagnetic signals and extracting tonic or phasic effects from the electromagnetic signal. The series of tests can include a series of information processing tasks, with information designed to be processed over varying periods of time. The electrical signals are preferably measured at multiple locations on the head of a patient and combined together. Further, the method can also include recording genetic, structural MRI and functional MRI information for the test patient.

### BRIEF DESCRIPTION OF THE FIGURES

Further features and advantages of the present invention will become apparent from the following detailed description of preferred embodiments of the invention, taken in combination with the appended drawings in which:

FIG. 1 is a schematic illustration of the interrelationship between sites and a main server;

FIG. 2 illustrated one form of experimental apparatus utilised at each site;

FIG. 3 illustrates the time continuum of tests provided in the preferred embodiment; and

FIG. 4 illustrates the corresponding tests that can be utilized.

FIG. 5 to FIG. 19 illustrate schematically screen shots of example exercises to be carried out by a user/patient;

FIG. 20 illustrates an example final report produced by the system.

### DESCRIPTION OF PREFERRED AND OTHER EMBODIMENTS

In the preferred embodiment, there is created a “International Brain Database” (IBD) which can be utilized by researchers as the “gold standard” for research in neuroscience. The present research takes an integrative approach as opposed to the largely myopic approach taken by research in the past.

The IBD establishes a normative database of subjects (nominally 1,000) that can be used as a reference population. 17 psychometric and psychophysiological tests used in the methods of the system of the preferred embodiment are designed to tap the brain's major networks. This system allows comparisons across these multiple tasks and comparison of subjects to the normative population.

The central analysis procedures of the IBD also introduce a number of new measures and methods. In particular, the role of gamma phase synchrony, and analysis of the tonic and phasic effects of arousal on cognitive measures. An additional element is the inclusion of a new brain modeling procedure, which enables an estimate of the basic neuro-physiological parameters for each individual. The normative nature of a database allows detection of specific enhancements and deficits when compared to the psychometric scores of an individual subject compared with previous systems.

To address the quality control and consistency issues of brain databases for medical purposes, the emphasis of the international database is on quality control measures such as identical set-ups and procedures in different laboratories to ensure comparability of data collected. A battery of psycho-physiological and psychometric tasks are used that are designed to tap many of the brains major cognitive networks. Central analysis of all the recorded data is undertaken, including the use of the new methods described below.

A significant benefit of the IBD of the present system is that the database provides access to normative data across a range of tests enabling the exploration of the interrelationships between Clinical Psychophysiology-Psychometric-Behavior and Demographic information.

The central nervous system can be likened unto a net. If you pull any single mesh in the met, the shape of every other mesh will change, which very aptly describes the relationship of the neurotransmitters in the brain. Rather than a single neurotransmitter being involved in the affective disorders, it appears that several may be important. The ratios of multiple neurotransmitters to one another may play a larger role than the actual amount present of any one single neurotransmitter.

The brief summary of some of the most common neurological disorders and the neurotransmitters that are generally accepted as being indicators of the respective disorders is provided hereinafter. The disorders addressed can include: Depression; Bipolar Disorder; Schizophrenia; Anxiety Disorders; Post-Traumatic Stress Disorder (PTSD); Attention Deficit Disorder (ADHD); Autism; Alzheimer's Disease; Closed Head Injury; Epileptic Disorders; and Parkinsons Disease.

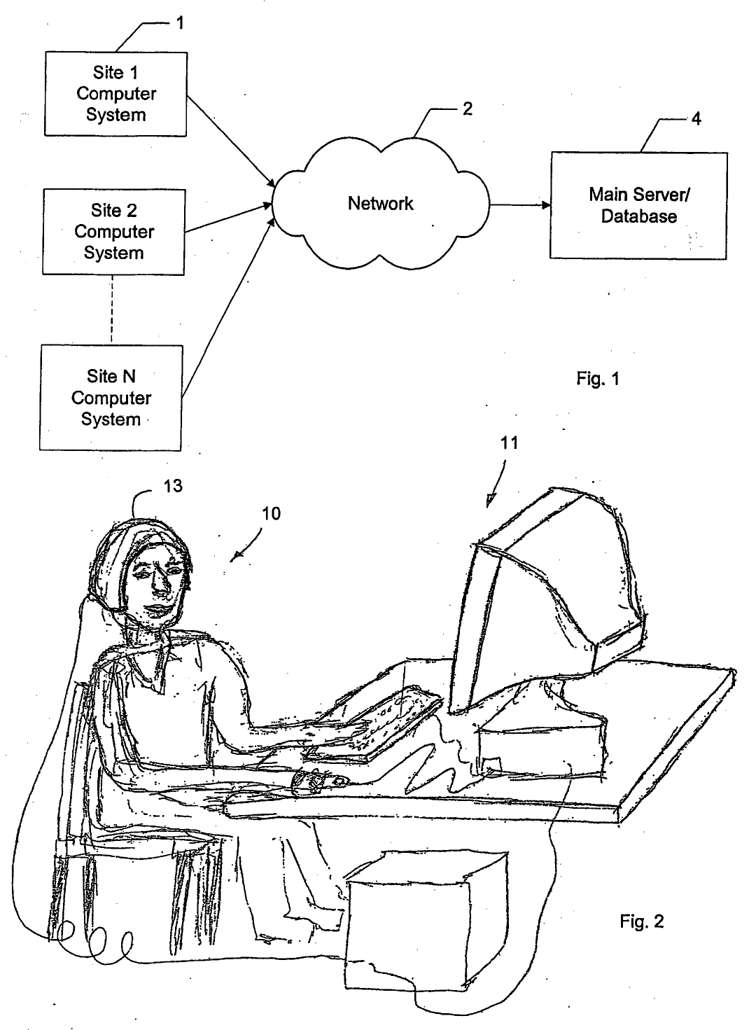

Turning initially to FIG. 1, the brain database consists of a series of sites e.g. 1 which are interconnected via a network 2 which can comprise the Internet to a server device 4 which collates the information from each site into an overall database. At each site e.g. 1 a patient is subjected to a series of tests and their brain waves measured. An example patient environment is illustrated in FIG. 2 wherein a patient or subject 10 interacts with a series of tests on a computer system 11. The computer system includes a touch screen interface. The subject 10 for one series of tests wears a monitoring device 13 which can comprise a 40 channel Nuamps or electrocap device (available from Compumedics USA Ltd) for measurement of electrical activity within the brain. This device can be interconnected with the computer system and provides for brain monitoring capabilities whilst the user 10 undertakes various tests.

Other measurements can be taken in addition to the EEG/ERP test provided by the device 13. These can include autonomic arousal (electrodermal, heart rate, respiratory rate, etc), MRI, genetics and psychological measurements. These tests are conducted on each subject with the results formatted in a standard form and sent to the main server.

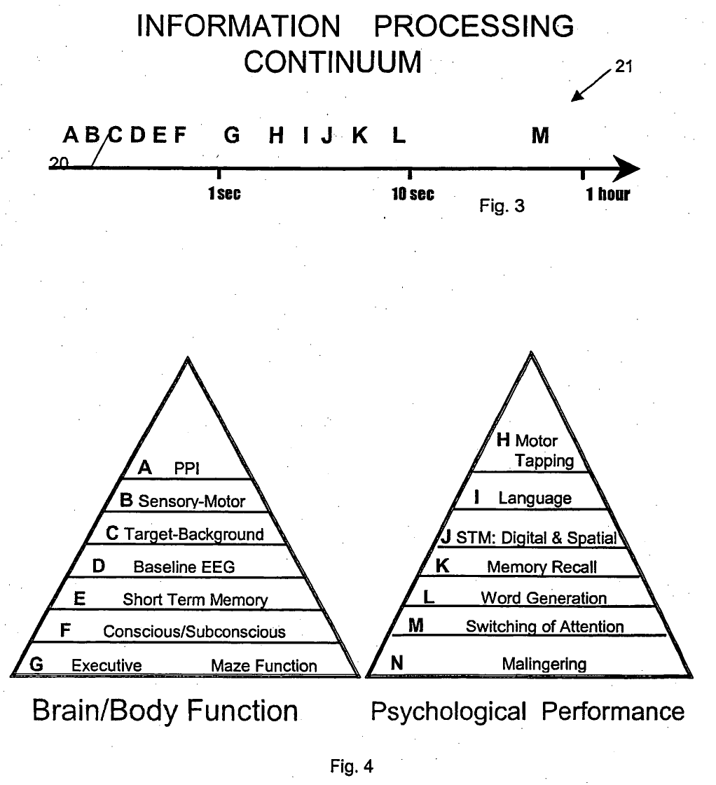

The tests can include various information processing tasks wherein information is designed to be processed over various periods of time. Turning to FIG. 3, there is illustrated a time line 20 having various example tests 21 denoted there along.

The tests can be drawn from the literature and can be directed at brain body function and psychological performance as illustrated in FIG. 4. Examples of suitable tests can be found in the book entitled “Integrative Neuroscience” by Dr. Evian Gordon, Harwood Academic Publishers 2000.

The tests provided can include an ongoing large range of standard and new tests. Indeed, new tests can be introduced on an ongoing basis so as to provide additional functionality.

In one example setup, the database includes tests relating to:

Activation Tasks; and

Psychological Tests.

Activation Tasks

The activation tasks relating to brain and body function are designed to tap the brain's major functional networks using the 40 channel Nuamps and Electrocap, including the following paradigms described hereinafter:

Resting EEG (cortical stability);

Habituation paradigm (novelty learning);

Auditory oddball (efficiency of target processing);

Visual tracking task (automatic tracking);

Go/No-Go (inhibition);

Conscious and subconscious processing of facial emotions;

Visual working memory task (memory and sustained attention);

Executive maze task (planning and error correction); and

Startle paradigm (fight and flight reflex).

These tests will now be described in more detail:

Resting EEG

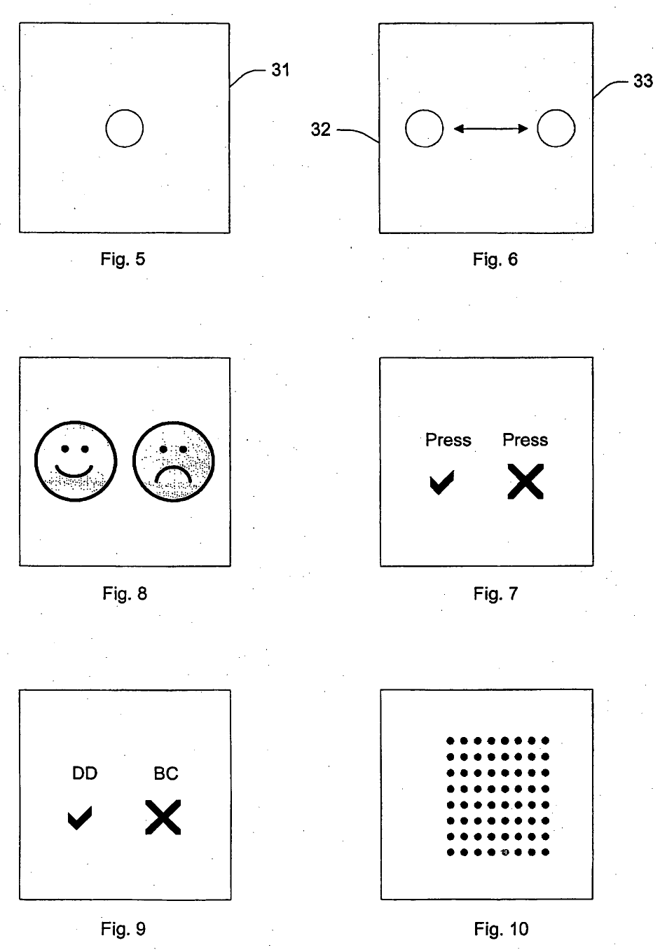

The subject is asked to rest quietly and focus on the red dot (eyes open) similar to that illustrated on the example screen 31 of FIG. 5 and then repeat the process with eyes closed. The task lasts for four minutes. The baseline EEG measure allows for comparison between resting and active states of the brain.

Test procedure: The subject is asked to rest quietly and focus on the red dot on the computer monitor 60 cm in front of them, with eyes open and then the paradigm is repeated with eyes closed.

Functions measured: The EEG primarily arises from the summation of electrical potentials in thousands of synchronously active dendrites in cortical neurons, particularly pyramidal cells which are lined in columns perpendicular to the cortical surface and their summated activity is thereby discernable.

EEG electrical currents are measured non-invasively using recording disks on the scalp and reflect synchronized and desynchronized operations of the overall cortical electrical activity (and their subcortical modulations) in the brain. The time resolution is in the order of seconds.

A small number of fundamental EEG rhythms (cycles per second or Hz) emerge and index the underlying stability of brain function and its general response to stimulation. These are as follows:

Delta: 0.5-3.5 Hz—This is best observed during deep sleep and is not generally prominent during cognitive activity;

Theta: 4.0 -7.5 Hz—This is also normally observed during sleep but also reflects aspects of learning and attention;

Alpha: 8-12 Hz—This reflects the idling state of the brain based on thalamocortical processing—a relaxed readiness. It diminishes (desynchronizes) with the level of brain activation;

Alpha peak frequency—This provides an index reflecting the capacity of verbal working memory;

Beta: >12 Hz—This increases with the level of brain activation;

Gamma: >35 Hz—Reflects integrative function across brain regions

The EEG exhibits transient states across these frequencies that are perturbed by stimuli, at which time they rapidly switch to a new transient state. The spatial distribution of the EEG power changes with these state changes. For example, with eyes closed, alpha is more evident at the back of the head than with eyes open and vice-versa for beta activity.

Each of these components can be measured in terms of their power (microvolts²) and their peak frequency. Power scores can be absolute (raw power for each frequency) or relative (each relative to the total power of all frequencies). The scores in these reports measure the amount of power exhibited by each of these frequencies during two resting conditions—one with eyes closed and the other with eyes open.

Putative brain regions involved:

Delta: Brain stem

Theta: Limbic system

Alpha: Thalamocortical

Beta and Gamma: Cortical

Neurotransmitters/receptors involved:

Delta: Activation of metabotropic glutamate receptor; GABA (A) receptor

Theta Cortex: noradrenergic neurotransmission; cholinergic neurons

Theta Hippocompal: serotonin inhibition; intrinsic noradrenergic activity; GABA interneurons

Alpha: Cholinergic (muscarinic receptors); GABA (B)

Beta: Nicotinic/cholinergic activation; GABA (A); dopamine

Gamma: GABAergic interneurons

Practical significance: Abnormal power in any or all of these fundamental frequencies reflects instability in brain function. However, changes in alpha and beta are also state dependent and the significance of the abnormality needs to be interpreted in conjunction with autonomic measures of arousal (sweat rate—skin conductance level [SCL] and heart rate that are simultaneously measured).

Changes in peak frequency of alpha, theta and delta are also often associated with brain pathology (structural or electrochemical).

EEG Scores

Average power spectra are computed for eyes open, eyes closed, and pre-stimulus target auditory oddball epochs. For eyes open and eyes closed paradigms, approximately two minutes of EEG are acquired. These two minutes of EEG are divided into adjacent intervals of four seconds. Power spectral analysis is performed on each four second interval by first applying a Welch window to the data, and then performing a Fast Fourier Transform (FFT). The resulting power spectra are averaged for each paradigm, yielding a single eyes open and a single eyes closed average power spectrum for each electrode position. For the pre-stimulus target auditory oddball epochs, the same procedure is followed, except that the epochs subject to power spectral analysis are only one second in duration, from one second prior to each target until the target presentation. Again, the pre-stimulus power spectra are averaged across targets, yielding a single pre-stimulus target auditory oddball power spectrum for each electrode.

For each average power spectra, the power is calculated in the four frequency bands, delta (1.5-3.5 Hz), theta (4-7.5 Hz), alpha (8-13 Hz), and beta (14.5-30 Hz). This power data is then square-root transformed in order that it might better approximate the normal distributional assumptions required by parametric statistical methods.

The data collected is then processed using an analytical numerical model. This mathematical model of the cortex incorporates realistic anatomical features, such as separate inhibitory and excitatory neural populations, range-dependent connectivities, dendritic delays, synaptic activation, firing thresholds, axonal conduction, nonlinearities, and both intra and intercortical pathways.

Global and local neural properties are represented by mathematical equations for the average firing rate of neurons within a macroscopic patch of cortex (cm²). Activity from excitatory and inhibitory cells produces post-synaptic potentials (PSPs), which are summated at the cell body. A sigmoid function relates firing rate to the potential at the cell body. Electrical activity then propagates away from the neurons in a (on average) concentric manner. The model equations can be combined to produce a single equation describing the EEG power spectrum, in terms of a small number of neurophysiological parameters.

The crucial neurophysiology of the brain is represented by parameters listed in the following table:

| Model | Parameter | Description | Initial Value |
|---|---|---|---|
| EEG | γₑ | Cortical damping (v/rₑ) | 130 s⁻¹ |
| Model | α | Dendritic decay rate | 75 s⁻¹ |
| | β/α | Dendritic response ratio | 3.8* |
| | t₀ | Conduction delay through | 0.084 s |
| | | thalamic nuclei and projections. | |
| | Gₑₑ | Excitory gain - pyramidal cells | 5.4 |
| | Gₑᵢ | Local intracortical gain - stellate cells | −7.0 |
| | Gₑₛₑ | Corticothalamocortical gain via SRN | 5.6 |
| | Gₑₛᵣₑ | Corticothalamocortical gain via TRN | −2.8 |
| | Gₛᵣₛ | Intrathalamic gain | −0.6 |
| | k₀rₑ | Volume conduction filter parameter | 3.0* |
| | lₓ, l_{y} | Linear dimensions of cortex | 0.5 m* |
| | rₑ | Characteristic pyramidal axon length | 0.08 m* |
| | P₀ | Overall power normalization | Calculated |
| | | | μV²/Hz |
| EMG | A | Power normalization | 0.5 μV²/Hz |
| | fₚₖ | Spectral peak frequency | 40 Hz* |
| | δ | Asymptotic slope | 2.0* |

Initial and fixed parameter values for the EEG and EMG theoretical model spectrum, are obtained from previous experimental work and standard references. All values are consistent with independent sources and physiological measures. The physiological estimates are consistent with experiment, as discussed in references and found during model fits to experimental data. In fixing certain model parameters the median value is observed.

To make use of the model a Levenberg-Marquardt method is used to adjust free parameters and fit its spectrum to EEG spectra. A maximum of 10 free parameters are adjusted; however for waking fits this is limited to seven by tying together cortical and thalamic dendritic time constants. Only in some sleep states do independent cortical and thalamic values appear necessary [see P A Robinson, C J Rennie, D L Rowe, S C O'Connor, Estimation of neurophysiological parameters on multiple spatial and temporal scales by EEG means: Consistency and complementarity versus independent measures, Human Brain Mapping 2004].

For the auditory habituation assessment the data analysis is concentrated on the skin conductance response (SCR) and the skin conductance level (SCL. The scoring of a phasic skin conductance response (SCR), to individual stimuli, is determined by measurement methods. SCRs elicited by stimuli are evaluated as an unambiguous increase in electrodermal activity (0.05 ms) with respect to each pre-stimulus baseline and whose initial rise occurs one to three seconds after a stimulus [see Barry R J, Sokolov E N. 1993, Habituation of phasic and tonic components of the orienting reflex, International Journal of Psychophysiology, 15 39-42].

For the auditory oddball assessment, analysis is performed on target and background ERP averages and gamma amplitude and synchrony waveforms.

Event-Related Brain Electrical Activity

The remainder of the activation tasks (habituation through to startle) were designed to measure electrical activity in the brain in response to a variety of stimuli. The Event-Related Potentials (ERPs) are transient electrical potentials occurring on a millisecond scale and which are time locked to discrete events (sensory stimuli or motor responses) during a task. Traditionally, EEG activity is sampled and time locked over multiple events of the same type and the samples averaged. This allows the extraction of brain activity that is specifically related to task processing i.e. the ERP.

The ERP generally consists of a series of peaks and troughs (components) that reflect stages of processing during task performance. The latency of these components reflects the speed of the related aspects of information processing. Component amplitude reflects the extent of cortical involvement in these processes.

Early components that occur with the first 80 ms following a stimulus mainly reflect obligatory processing by the brain to external events. They are routinely used to reflect the integrity of sensory neural pathways. Later components are primarily associated with task-related processes, such as:

Obligatory and early attentional processing to stimuli (N100-P200).

N200-P300 reflects processing due to novelty, orienting and significance evaluation of stimuli. There are multiple types of N200-P300 componentry, including the ones scored in this report.
i. Detection of stimulus change (N200)
ii. Face perception (N170)
iii. Orienting (P300a)
iv. Assessment of contextual significance (P300b)
v. Updating of verbal working memory (P450)
vi. Detection of contextual incongruity and semantic elaboration (N400)

Integrative processing of these activities is reflected by gamma phase synchrony.

The components assessed in each of the above tests are:

1) Auditory oddball:
i. Target processing—N100, P200, N200, P300b
ii. Backgrounds—N100, P200

2) Visual working memory:
i. Backgrounds: P450

3) Go-No Go task: N200

4) Processing of facial emotions: N170, VPP, P300a

The putative brain regions involved are:

1) N100—networks involving the secondary sensory cortices

2) N170—temporal cortex

3) P200 (or VPP, vertex positive potential)—visual association cortices and medial frontal regions

4) N200—frontal cortices

5) P300a—anterior cingulate region of frontal cortex

6) P300b—hippocampus and temporo-parietal association cortex

7) N400—left hemisphere, anterior temporal and lateral prefrontal cortices

8) P450—parietal association cortex

The neurotransmitters/receptors involved are:

1) P100—cholinergic neuronal projection system

2) N100—GABA (A) receptor, GABA

3) P200—alpha2 noradrenergic receptor

4) N200—GABA, Dopamine

5) P300—cholinergic, noradrenergic, dopaminergic, serotoninergic and gabaergic systems.

That is, more specific fast acting neurotransmitters are involved for early components (in particular, cholinergic, GABA and noradrenalin), but it is a combination of interacting neurotransmitters (including slow acting ones such as serotonin and dopamine) for later components.

The practical significance of the ERPs is to test a range of aspects of sensory, motor and cognitive activity by the brain. A fundamental distinction between ERPs and the Cognitive Performance profile rests in the time domain. ERPs provide real time indices of neuropsychological processes, on the time scale of milliseconds, whereas the measures obtained in the Cognitive Performance profile represent the behavioral outcomes of such processes, on the time scale of seconds.

ERPs provide the highest temporal resolution of brain imaging technologies and are therefore used as real time, biological markers of both psychological and physiological events in the brain.

Abnormalities in such components (amplitude or latency) respectively reflect dysfunction in the brain's contribution to these processes or in processing speed.

The abilities assessed in each paradigm are broken down as a function of each of the tests as follows.

Habituation

Subjects are instructed to look at a red dot on the screen (as illustrated in FIG. 5). They are told they will hear some sounds, but just to ignore them. Ten tones (500 Hz) are presented at a 1 s inter-stimulus intervals (ISI), followed by a change stimulus (1000 Hz) and then five repeats of the initial tones (500 Hz). This task lasts for one minute.

Auditory Oddball

Subjects are instructed to look at a red dot on the screen (as illustrated in FIG. 5). Subjects are presented with a series of high and low tones, at 75 dB and lasting for 50 ms, with an ISI of 1 s. The rise and fall times of the tones is 5 ms. Subjects are instructed to press buttons with the index finger of each hand in response to ‘target’ tones (presented at 1000 Hz). They are asked not to respond to ‘background’ tones (presented at 500 Hz). Speed and accuracy of their response is equally stressed in the task instructions. The background and target tones are presented in a quasi-random order, with the only constraint being that two targets cannot appear consecutively. The duration of the auditory oddball task is six minutes. This task allows for assessment of basic sensory-motor and decision-making mechanisms.

Visual Tracking

As illustrated in FIG. 6, subjects are instructed to follow a red dot 32-33 with their eyes as it moves across the screen at 0.4 Hz, but not to move their head. The task lasts for one minute.

Go/No-Go Task

As illustrated in FIG. 7, subjects are repeatedly presented with the word ‘PRESS’ (for 500 ms) on the screen in front of them, with an ISI of 1 s. If the word appears in red, the subject is asked to do nothing. If the word appears in green, the subject is asked to press buttons with the index finger of each hand. Speed and accuracy of response are equally stressed in the task instructions. The word ‘PRESS’ is presented in the same color 6 times in a row. There are 28 sequences, 21 of which are presented in green and 7 in red, presented in a pseudo-random order. The duration of the go-no go task lasts for approximately 5 minutes. This task tests the executive functions of the pre-frontal cortex, in particular its ability to inhibit inappropriate motor responses.

Processing of Facial Emotions

Unconscious: Subjects are told they will see a series of different faces such as those shown in FIG. 8, presented in pairs, but that the first face of each pair will be presented so briefly as to be barely visible. They are told they do not need to anything but sit still, but that they need to pay attention, as they will be asked about the faces later on.

Conscious: Subjects are told that they will see a different series of faces, but that these will be presented only one at a time. Again, they are instructed to sit and relax, but to pay attention to the faces because they will be asked about them subsequently The total task time for face stimuli is 11 minutes.

Visual Working Memory

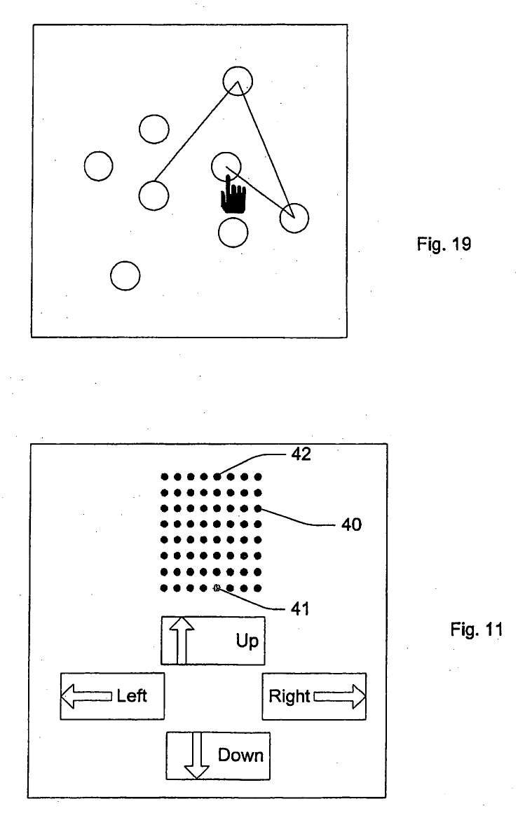

This task consists of a series of letters (B, C, D or G) presented to the subject on the computer screen (for 200 ms), separated by an interval of 2.5 seconds. If the same letter appears twice in a row, the subject is asked to press buttons with the index finger of each hand. Example letters are shown in FIG. 9. Speed and accuracy of response are equally stressed in the task instructions. There are 125 stimuli presented in total, 85 being non-target letters and 20 being target letters (i.e. repetitions of the previous letter). The task is designed to assess basic memory processes. The remaining 20 stimuli are checkerboard patterns similar to that shown in FIG. 10 with black and white squares, 1 cm in width. This latter stimulus elicits the P300a visual ERP, which is a measure of the processing of novelty. The checkerboard never occurs immediately preceding a target letter (by definition). This task lasts for approximately six minutes.

Executive Maze

As shown in FIG. 11, subjects are presented with a grid (8×8 matrix) of circles 40 on the computer screen. The object of the task is to find the hidden path through the grid, from the beginning point at the bottom of the grid 41 to the end point 42 at the top. The subject is able to navigate around the grid by pressing arrow keys e.g. 43. The subject is presented with one tone (and a red cross at the bottom of the screen) if they make an incorrect move, and a different tone (and a green tick at the bottom of the screen) if they make a correct move. The maze is the same each time the subject does the task. The purpose of the task is therefore to assess how quickly the subject learns the route through the maze and their ability to remember that route. When the subject makes their way through the maze twice, without making any mistakes, the trial ends. Since the task requires coordination of visual, motor and memory skills, it can be used to assess executive function. The duration of the maze task is eight minutes maximum.

Startle

The subject is asked to sit comfortably in the chair and fixate on a red dot (FIG. 5) on the computer screen, ignoring any sounds they might hear. The subject is then presented with a series of acoustic startles (noise burst of 50 ms at 100 dB, instantaneous rise and fall). This sound is designed to elicit the startle response, which consists primarily of the eye-blink reflex. This reflex is measured by recording the muscle activity around the eye. Successive stimuli are separated by a random interval between 10 and 15 seconds. Some startle stimuli can be preceded by 50 ms with a pre-pulse, which consists of quieter noise burst (20 ms at 75 dB with a 5 ms rise and fall time). This pre-pulse has the effect of inhibiting the startle response, and can be used to measure sensory gating mechanisms in the subject. This task lasts for approximately four minutes.

ERP Scores

Average ERPs are calculated for (a) target and background auditory oddball stimuli, (b) target, background and checkerboard (distracter) visual working memory stimuli, (c) go and no-go stimuli in the visual go/no-go paradigm, (d) conscious and unconscious faces stimuli for each of six emotions (neutral, happy, fear, anger, disgust, sadness). In each of these cases the individual single-trial epochs were filtered with a low-pass Tukey (cosine tapered) filter function that attenuates frequencies above 25 Hz. The single-trials are then averaged to form conventional ERPs. Peak identification and difference waveform analysis can also be incorporated as required.

For the emotional faces, difference waveforms were also formed for each of the five emotions. This involves subtracting the emotion face ERP from the neutral face ERP in the same condition (i.e. conscious or unconscious). This results in five ERP difference waveforms: fear-neutral, happy-neutral, anger-neutral, disgust-neutral, and sadness-neutral, for each of the two conditions, conscious and unconscious.

Gamma phase synchrony: the measure of the phase synchronization of Gamma activity is assessed across multiple brain regions in humans. Before Gamma phase synchrony analysis, all single-trials have any linear trend in the time domain removed by subtracting the line of best fit over 1024 samples (2.048 s) centered at the stimulus presentation. For each single-trial waveform a 128 sample Welch window is moved along sample by sample, starting with the center of the Welch window at 500 ms prior to the stimulus (-500 ms) and ending with the center of the Welch window at 750 ms after the stimulus. At each sample position, the phase of the Gamma frequency component is computed by means of FFT, yielding a time series of Gamma phase from −500 to 750 ms for each single-trial from each site. Since the sampling rate is 500 Hz and the window length is 128 samples, the width of each frequency bin is 500/128 or 3.91 Hz. Thus there is a bin centered at 39.1 Hz, which extended from 37.1 Hz to 41.0 Hz, or approximately 37 to 41 Hz, which is the primary bin analyzed.

Following this calculation of the time series of gamma (37 to 41 Hz) phase for each site, the phase synchrony across sites within various regions of interest is calculated at each sample point in time from −500 to 750 ms. Phase synchrony is defined to be the inverse of the circular variance of phase across sites. Circular variance is an index of the extent to which the sites are in phase or phase-locked with each other. Circular variance is a normalized measure that ranges from 0 to 1 and is completely independent of the amplitude of the responses. Like a correlation coefficient, it therefore has no associated units of measure. It can be thought of as similar to a coherence estimate, except that it is an index of the extent of phase-locking across many sites rather than just between two sites as with coherence. For ease of interpretation phase synchrony is calculated as the inverse of circular variance, or simply one minus the circular variance.

This analysis produces a time series of values which represent (in units of circular variance) the extent of phase-locking (or how homogenous the phases are) for Gamma activity, as a function of time, within the sites making up each region of interest. The regions of interest will vary with the task. However, the core regions of interest, in addition to global (all sites), synchrony are. frontal (Fp1, Fp2, Fz, F3, F4, F7 and F8), centro-temporal (T3, C3, Cz, C4 and T4), fronto-central (F3, Fz, F4, FC3, FCz, FC4), parieto-occipital (Pz, P3, P4, O1, Oz and O2), and posterior (CP3, CP4, T5, P3, Pz, P4, T6, O1, Oz and O2) as well as waveforms to examine lateralization, these being left hemisphere (Fp1, F3, F7, FC3, C3, CP3, T3, T5, P3 and O1), midline (Fz, FCz, Cz, CPz, Pz and Oz) right hemisphere (Fp2, F4, F8, FC4, C4, T4, CP4, P4, T6, and O2), left centro-temporal ( T3 and C3) and right centro-temporal (C4 and T4) and waveforms to examine quadrants effects, these being right frontal (Fp2, F4, F8 and FC4), left frontal (Fp1, F3, F7and FC3), right posterior (CP4, P4, T6 and O2) and left posterior (CP3, T5, P3 and O1). Within each of these regions, the extent of Gamma phase-locking (‘phase synchrony’) is examined (Symond M, Harris A W F, Gordon E & Williams L M. (2005). Gamma synchrony” in first-episode schizophrenia: a disturbance of high temporal-resolution functional connectivity. American Journal of Psychiatry, 162, 459-465; Williams L M, Grieve S, Whifford T J, Clark C R, Gur R C, Goldberg E, Peduto A S, Gordon E. (2005). Neural synchrony and gray matter variation in human males and females: an integration of 40 Hz gamma synchrony and MRI measures. Journal of Integrative Neuroscience (in press); Paul R H, Clark R C, Lawrence J, Goldberg E, Williams L M, Cooper N, Cohen R A, Gordon E. (2005). Age-dependent change in executive function and gamma 40 Hz phase synchrony. Journal of Integrative Neuroscience (in press).

These phase synchrony waveforms must necessarily be computed at a single epoch level, so the waveforms from the epochs of interest (the same as those listed for conventional ERPs) are then averaged for each subject in the same manner as for conventional ERPs. Each average synchrony waveform is then smoothed with a 15 point running average.

Following this, the area under the curve (total synchrony) is calculated for the time windows −100 to 150 ms and 200 to 450 ms. Synchrony is calculated relative to a −450 to −150 ms pre-stimulus baseline average.

Data analysis of the various tests can also include:

Eye-tracking
Pre and post-stimulus power spectra (see above)
Analytical model (see above)
SCR/SCL (see above)
Target reaction time

Go/no-go
Red and green ERP averages.
Gamma amplitude and synchrony waveforms.
Pre and post-stimulus power spectra.
Analytical model.
SCR/SCL.
Green reaction time.

Passive letter viewing
Data analysis: ERP averages.
Gamma synchrony and amplitude averages.

Working memory task
ERP averages (early language processing is reflected by P90-N150 at P3 and P4 sites; updating of working memory is reflected in P440-550 at Fz and Pz sites).
Gamma synchrony and amplitude averages.

Executive maze function
Behavioral measures, e.g. number of overruns.
ERP and EEG.

For the EEG analysis, each 2 minute recording (for eyes closed and for eyes open) is divided into 2 second epochs, and power spectral estimation performed for each epoch at each recording site by applying a Welch window and then Fast Fourier Transformed (FFT) to the signal. The power spectra are then averaged for each recording (eyes closed, eyes opened) at each recording site. The following total power scores are derived at each site (all power values are square root transformed before statistical analysis):

Delta: 1.5-3.5 Hz.

Theta: 4-7.5 Hz.

Alpha: 8-13 Hz.

Beta: 14.5-30 Hz.

Also recorded is the Alpha peak frequency which is the maximum peak in the EEG spectrum that occurs between 8 and 12 Hz and the Alpha peak power at the peak frequency in the alpha (8-13 Hz) range. The Alpha peak frequency is best measured when subjects are resting with their eyes closed.

Since all these measures exist for each of the 26 scalp sites, a multivariate statistical comparison (Mahalanobis distance) is performed between the client and the controls.

For the ERP analysis, conventional ERP averages are formed at each recording site. Before averaging, each single-trial waveform is filtered at 25 Hz with a Tukey or cosine taper to 35 Hz, above which frequency no signal is passed. Waveforms are produced for each stimulus of interest for the task (eg. for processing of task-relevant ‘target’ stimuli during an ‘oddball’ cognitive task). For each stimulus of interest, the ERP components elicited by these stimuli (eg. N100, P200, N200 and P300 components for the oddball task), are identified relative to a pre-stimulus baseline average of −300 to 0 ms. The peak amplitude and latency is quantified for each component at each of the 26 recording sites. A multivariate discriminant analysis (using Mahalanobis distance) is performedto compare between the client and the control/peers. This comparison is based only on peer controls who are closely matched to the client in age, gender and years of education.

For each epoch, the gamma (37 to 41 Hz) phase synchrony is computed as a function of time within 7 regions of interest. The phase synchrony waveform for a given region and epoch is computed as follows. Firstly, time series (from −500 to 750 ms) of the phase of gamma oscillations were derived for each site in the region by means of a moving Welch window and short-time FFT. Then the circular variance of phase is computed across the sites in the region for each point in time. Once the synchrony waveforms are computed, the waveforms from all the target epochs from a given region are averaged, and similarly for all the background epochs from that region. This yields a single average target synchrony waveform, and a single average background synchrony waveform, for each region. A pre-stimulus baseline synchrony average (from −450 to −150 ms) is then subtracted from the waveform and the waveform is inverted for ease of interpretation. The 7 regions used are all sites: global, frontal, centro-temporal, parieto-occipital, left hemisphere, midline, and right hemisphere. Two measures (Gamma1 and Gamma2) exists for each of the 7 regions, therefore a multivariate comparison (Mahalanobis distance) is performed between the client and the controls. The Gamma1 measurement is the total synchrony (area under the curve) for the latency window −100 to 150 ms. The Gamma2 measurement is the total synchrony (area under the curve) for the latency window 200 to 450 ms.

Psychological Tests

The psychological tests include measures of attention, memory, personality dimensions and executive function. This allows for covariance with the brain measures and also tests for relationships between a wide array of these variables.

The Psychological Tests can serve to explore a profile of: Sensory-Motor, Language, Attention, Memory and Executive functions. These tests can be administered using the computer-based system of FIG. 2 and employing pre-recorded spoken task instructions. A touch screen interface can be used to allow direct touch to screen responses in addition to the recording of sound files for tests requiring an oral response.

The tests can include:

Motor tapping (motor coordination);

Choice reaction time (speed of motor reflex);

Timing test (capacity to assess time);

Digit span (short term memory);

Memory Recall and Recognition (Words repeated 5 times with a matched distracter list after trial 4);

Spot The Real Word Test (word: non-word index of IQ);

Span of Visual Memory Test (4 second delay test of spatial short term spatial memory);

Word Generation Test (Verbal fluency test);

Verbal Interference Test (test of inhibitory function);

Sustained Attention Test (ability to sustain attention to a task);

Switching of Attention (alternation between numbers and letters);

Executive Maze; and

Malingering Test (number recognition malingering test).

Motor Tapping Test

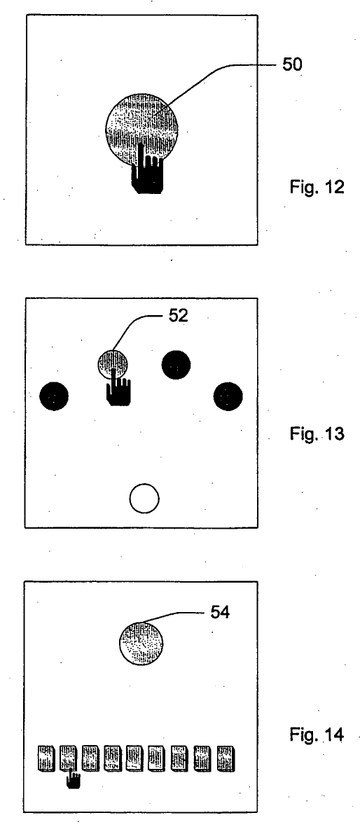

The motor tapping test, illustrated schematically in FIG. 12, requires the subject to tap a circle 50 on the touch-screen with their index finger as many times as possible in thirty seconds. The test is repeated for both hands. The purpose of the test is to assess basic hand-eye coordination since many of the tests require a similar response. Basic hand-eye coordination can then be factored into the results of the other tests, using statistical techniques. The finger tapping test is also a method of picking up the early symptoms of various types of movement disorders, such as Parkinson's disease, though its specificity would be poor.

Test procedure: The subject is required to tap a circle with the index finger of each hand in turn, as fast as possible.

Functions measured: Hand eye coordination and fine movement speed (manual dexterity).

Putative brain regions involved: Motor cortex, basal ganglia and cerebellum

Practical significance: Everyday motor skills such as typing and machine operation

Scores recorded:

Number of taps (the number of times the subject tapped the touch screen within 30 seconds with their right or left hand); and

Tapping Variability (the standard deviation between taps).

Choice Reaction Time Test

In a choice reaction-time test, subjects are given a stimulus, from a set of possible stimuli, and then have to match that stimulus to the appropriate response from a number of possible responses. In the version of the test used, as illustrated in FIG. 13, one of four circles 52 lights up, in different positions on the touch-screen. Immediately following presentation of the lighted circle, the subject has to touch that circle as quickly as possible. There are 20 trials in this task, and there is a random delay between trials of 2-4 seconds. The task takes approximately three minutes. The choice reaction-time test helps assess basic sensory-motor functions. Psychologists break choice reaction-time tasks like this into three separate stages of cognitive processing. In the first stage stimuli have to be identified, and in our version of the test this is simply spatial location. In the second stage, stimulus identification has to be mapped to the appropriate response; in this test the relationship between stimulus and response is very straightforward but never-the-less a translation between the sensory and motor systems is still required. In the third stage, motor responses are mobilized. Of course, all three ‘stages’ of processing can occur sequentially or in parallel, and the types of errors that a subject makes give clues as to the type of strategy they are pursuing in the task.

Test procedure: One of four circles lights up and the subject is required to press the lit circle as quickly as possible.

Functions measured: Visuomotor coordination, speed and accuracy of selecting an appropriate response.

Putative brain regions involved: Occipital, parietal, frontal and motor cortices, diencephalon.

Practical significance: Visual discriminative judgment and response. Examples: visual monitoring tasks requiring choice and reaction such as air traffic control, driving judgment.

Scores recorded:

Reaction Time (the average time that the subject took to tap a lit circle).

Timing Test

This test, illustrated in FIG. 14, assesses the subjects capacity to assess time. A circle 54 appears on the screen for 1 to 12 seconds, then the subject is required to indicate the correct duration of the circle's appearance by pressing a corresponding square 55.

Test procedure: A circle appears on the screen for 1 to 12 seconds and the subject is required to indicate the correct duration.

Functions measured: Ability to accurately estimate time duration.

Putative brain regions involved: Hippocampus and cerebellum.

Practical significance: Time organization.

Scores recorded: Proportional Bias: The value of the average difference between the actual length of the stimulus(l_{S}) and the subject's estimate(l_{U}) weighted by the length of the stimulus, i.e.:

$$abs ( \sum \frac{1}{n} \frac{{l}_{u} - {l}_{s}}{{l}_{s}} )$$

Span of Visual Memory Test

The span of visual memory test, as the name suggests, assesses spatial short term memory abilities on a visual task. As illustrated in FIG. 15, nine squares on the touch-screen light up in a random order. After a four second delay, the subject hears a tone indicating they have to reproduce, by pressing the squares, the order in which the squares lit up. In the psychological literature, this is called a delayed matching-to-sample test. This test assesses aspects of working memory. These aspects include the capacity to hold and sequence visuo-spatial information in short term memory.

Such memory is used in the everyday environment when a person has to remember, for a short period of time, some piece of information about their environment whose significance may or may not yet be known. A simple example is momentarily remembering the spot on the supermarket shelf where you took the coffee beans, in case you decide to swap it for another brand. The information is not stored in long-term memory as a ‘fact’ because it is not likely to be relevant by the next time you shop. Instead, a short-term memory representation is formed, utilizing a temporary network of electrochemical activity in the brain. Performance on such tasks improves with age until young adult-hood, and slowly declines thereafter. Imaging studies have shown that the pre-frontal and frontal lobes of the cortex are important to the retention of short-term memory.

Test procedure: The subject is required to press a series of squares on the screen in the order in which they previously lit up.

Functions measured: Short term visuo-spatial memory and attention.

Putative brain regions involved: Parietal, motor and prefrontal cortex.

Practical significance: ability to hold and retain new spatial information. A skill crucial to most everyday, non verbal tasks requiring memory. Examples include navigation, operating industrial machines.

Score: Length of the longest sequence correctly identified twice.

Digit Span Test

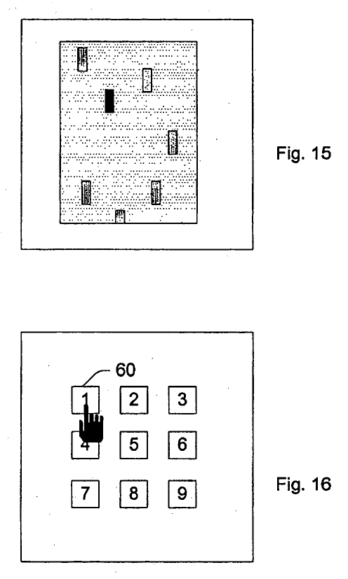

This is a test to assess the subject's short term memory function. The subject hears a series of digits (4, 2, 7 etc., 500 ms presentation), separated by a one second interval. The subject is then immediately asked to enter the digits, as illustrated on FIG. 16, into a on a numeric keypad 60 on the touch-screen, either in forward order or backwards (Reverse Digit Span task). The number of digits in each sequence is gradually increased from 3 to 9. The score on this test is given by the maximum number of digits the subject can reliably repeat without making mistakes. The digit span test taps one of the basic capacities of the short-term memory system. People are able to store only a limited number of simple items in their short-term memory. This is referred to as ‘seven plus or minus two’, since seven is the number of items a person of average ability can hold in memory and five to nine is roughly the range of ability in the population. An example of this effect is the ability to hold someone's birthday in short-term memory (4-4-65) for a short period of time without repeating it to yourself. On the other hand, an unfamiliar 8 digit telephone number would have to be repeatedly rehearsed by most people in order to remember it. This task takes approximately 5 minutes.

Test procedure: The subject is presented with a sequence of digits and then has to repeat them in either forward or backward order.

Functions measured: Short term verbal memory, working memory operations.

Putative brain regions involved: Prefrontal, temporal and inferior parietal cortex.

Practical significance: Ability to hold, retain and operate on new verbal information. A skill crucial to most everyday, verbal tasks requiring memory. Everyday examples include remembering telephone numbers and shopping lists.

Scores recorded:

Length of the longest sequence correctly recalled in forward or reverse order;

Ratio (the ratio of the score in forward and reverse order); and

Difference (the difference of the score in forward and reverse order).

Memory Recall and Recognition Test

The first part of this test is a memory recall task, which assesses the verbal memory of the subject. The subject is presented with a list of 12 words, which they are asked to memorize. The list contains 12 concrete words from the English language. Words are closely matched on concreteness, number of letters and frequency. The list is presented 4 times in total and the subject is required to recall as many words as possible after each presentation. Answers are recorded through a microphone into ‘.wav’ files. The subject is then presented with a list of distracter words and asked to recall those. The subject is then asked to recall the 12 words from the original list. This task takes approximately six minutes. Twenty-five minutes later, the subject is again asked to recall the 12 words from the original list. In the second part of the test, the subject's recognition of the previously presented words is tested. The subject is presented with a series of words on the screen (on some of which appeared in the original list) and asked to respond ‘yes’ or ‘no’ as to whether the word was in the original list of 12. Finally, each of the words in the different lists is presented on the screen and the subject is required to repeat the word out loud. This tests the subject's basic pronunciation ability. This second part of the test takes approximately four minutes.

Test procedure: The subject is asked to recall a set of words after various time intervals and later recognize the words from a list of repeated and new words.

Functions measured: Ability for new auditory verbal learning, memory recall and recognition. Verbal self-monitoring.

Putative brain regions involved: Involvement of fronto-parietal networks, including premotor, left prefrontal, left precuneus and left parietal regions.

Practical significance: Ability to learn and remember new tasks based on verbal information.

Scores recorded:

Score Trial n (the number of words correctly recalled within 30 seconds in trial n. Repeated words are counted only once);

Total Score Trials 1-4 (the sum of the scores in trials 1, 2, 3 and 4);

Total Intrusions Trials 1-4 (the number of times a word not in the list was recalled in trials 1-4);

Total Repeats Trials 1-4 (the number of times a word was repeated in trials 1-4);

Score Trial 5—Distractor List (the score for the words recalled from the new list used in the fifth trial);

Score Trial 6 (the number of words recalled from the first list—after the recall of the distracter list);

Score Trial 7—Delayed Recall (the number of words recalled approximately 40 minutes after trials 1-6); and

Learning rate (the slope of the linear regression of the scores in trials 1-4).

Recognition Scores:

Recognition Accuracy (the number of words from the memory recall list that were correctly recognized); and

Rejection Accuracy (the number of words that where correctly rejected as not being in the memory recall list.

Verbal Interference Test

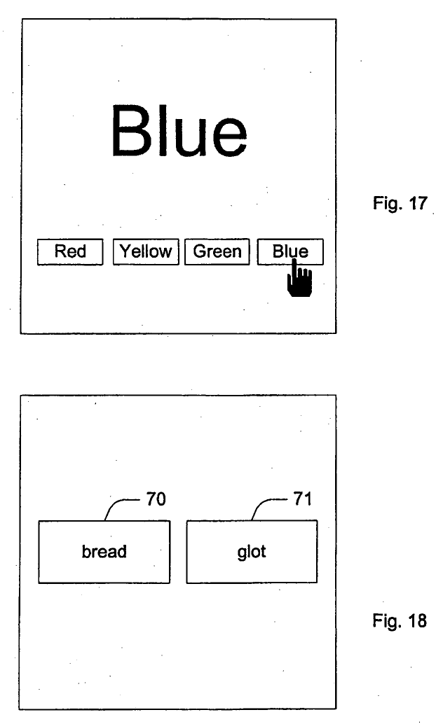

This is a test of inhibitory function and is made up of two sections. In the first section, the subject is required to indicate the color that the written word spells (and not the incongruent ink color that the word is written in). In the second section, the subject is asked to name the ‘ink’ color a word is written in (and not read the actual word). The verbal interference test is based on a similar test in the psychological literature, known as the ‘Stroop’ test after its creator. There are various versions of the test, but the core test, as illustrated in FIG. 17, involves the presentation of words describing colors, for example ‘green’, ‘blue’ and ‘red’. The words are written using colors which are different to the color described by the word, for example the word ‘green’ written in a red typeface. Subjects are asked to name the color of the ‘ink’ and ignore the written word. This is a surprisingly difficult thing to do at speed, and reaction time is used as a measure of performance. The ‘interference’ experienced from the written word is called the ‘Stroop’ effect. The interference arises from the fact that reading is a highly over-learned skill and occurs automatically unless there is a sustained attentional focus to suppress the reading response. Other versions of the test, without colored ink, or using colored patches instead of words, can be used to assess reading skill and color recognition, ruling out these influences as factors in test results.

The Stroop test is a highly sensitive measure of early dementia and frontal brain damage, though it may not be specific as an indicator of these problems.

Test procedure: The subject is required to name the ink color that a word is written in, and not the actual word.

Functions measured: Ability to inhibit inappropriate well-learned impulsive automatic responses.

Putative brain regions involved: Multiple cortical sites mediated by the anterior cingulate cortex.

Practical significance: Ability to control impulses; behavioral control e.g. anger control.

Scores recorded:

The number of correct responses in recognizing the color or the text of the displayed word;

Errors (the number of incorrect responses);

Reaction Time (the average time to identify a stimulus when the response was correct);

Score(color)−Score(text) (the difference between the second and the first task scores);

Errors(color)−Errors(text) (the difference between the second and the first task errors);

RT(color)−RT(text) (the difference of the average reaction times between the second and the first task);

Score(color)/Score(text) (the ratio of the scores in the second and the first task);

Errors(color)/Errors(text) (the ratio of the errors in the second and the first task); and

RT(color)/RT(text) (the ratio of the average reaction times in the second and the first task.

Spot The Real Word Test

An important tool in neuropsychological assessment is the ability to estimate the intelligence of a subject before onset of their particular disorder or disease. This is called ‘pre-morbid IQ’. For obvious reasons, tests of intelligence prior to disease onset are not commonly available. This test enables an estimate of pre-morbid IQ to be made, which can then be compared to measures of current intelligence to assess the impact and time-course of the disorder or disease. The test, illustrated in FIG. 18, consists of a word 70 and nonsense 71 word pair presented on the touch-screen. The subject has to indicate which is the ‘real’ word by pressing the touch-screen.

This test is thought to be particularly resilient to various forms of brain dysfunction and damage because it is a task that can be performed using many different strategies. Words can be distinguished from non-words on the basis of rote recognition, their general familiarity, their meaning, their orthographic appearance (visual shape), or their sound (when vocalized internally). One or more of these routes may be blocked by various brain disorders, but the other routes tend to remain independently functional and so can be utilized by the subject to reveal their otherwise hidden word knowledge.

Test procedure: A real word is presented simultaneously with a nonsense word. The subject is required to select the real word.

Functions measured: English language recognition.

Putative brain regions involved: Broad cortical involvement but particularly left perisylvian regions (e.g. Wernickes area).

Practical significance: language skill; correlates with premorbid intelligence.

Score: Number of words correctly recognized.

Word Generation Test (Verbal Fluency Test)

The word generation test is designed to measure verbal fluency, or an individual's capacity to produce a sustained stream of spontaneous speech. The test involves the subject naming as many words as possible, in the space of a minute, which begin with a certain letter. Subjects are instructed not to use proper nouns, nor to make variations on the same word stem (‘run’ and ‘running’ for example). The letters most commonly used in the test are F, A and S, for which word naming is relatively easy. The score on the test is simply the number of words produced for each of the three letters.

Brain imaging studies have shown that left frontal areas are critically involved in this task. The test is particularly sensitive to traumatic brain injury involving the frontal or temporal lobes or the caudate nucleus. Ability on the word generation test is modified by years of education and ethnic origin, but less so by age and is uninfluenced by gender.

Test procedure: The subject is required to say as many words as possible (in 1 minute) which start with given letters and then state as many animals as possible.

Functions measured: Verbal fluency and thinking ability.

Putative brain regions involved: Include left inferior frontal cortex, left dorsolateral prefrontal cortex, supplementary motor cortex, the anterior cingulate cortex and the cerebellum.

Practical significance: Ability to generate and articulate thoughts and ideas in a systematic manner.

Scores recorded:

FAS Score (the average number of words generated in one minute that began with a specific letter); and

Animal Score (the number of animal words generated in one minute).

Sustained Attention Test

This test assesses the ability to sustain attention over an extended period on a task involving a sequence of letters presented one at a time on the visual display monitor and short-term memory. The task is to detect occasional target letters embedded in the stream of letters presented. A target letter is defined as a letter that is the same as a preceding letter. The subject is asked to press a button if the same letter appears twice in a row. Thus, successful performance requires remembering each letter as it comes up for comparison with the next letter. In this way, the test assesses the ability to update information held in the verbal short term stores of working memory. This ability is reflected in the number of targets correctly detected. Novel stimuli are also presented.

Test procedure: The subject is presented with letters one by one, pressing a button if the same letter appears twice in a row.

Functions measured: Sustained attention, target detection.

Putative brain regions involved: Dorsolateral prefrontal and medial frontal cortex, thalamus, basal ganglia, posterior parietal and superior temporal lobe.

Practical significance: Ability to detect and respond to significant change under conditions requiring vigilance. Fundamental everyday skills e.g. train, plane, automobile, computer and equivalent machine operations.

Scores recorded:

Reaction Time (the average reaction time to identify the repeated letters);

False alarm rate (the number of incorrect responses); and

Missed targets (the number of targets that the subject did not respond to).

Switching of Attention

This test contains two simple tests of attention. The first requires the connecting of numbers in ascending sequence (i.e. 1-2-3-etc). As illustrated in FIG. 19, 25 numbers, in circles, are placed on the touch-screen and the subject has to press them in the correct order. This tests the basic ability to hold attention on a simple task. The second requires the connecting of numbers and letters in ascending but alternating sequence (i.e. 1-A-2-B etc). The numbers 1-13 and the letters A-L are presented in circles on the touch-screen. This tests the ability to alternate attention between simple mental sets. This task has a four minute duration.

Test procedure: Numbers and letters are connected up sequentially in chronological order.

Functions measured: Visuomotor tracking, attention, ability to shift the course of ongoing mental activity.

Putative brain regions involved: Dorsolateral frontal cortex.

Practical significance: Ability to sustain and control the direction of attention. Critical activity for everyday to multitasking skills e.g. management, driving.

Scores recorded:

Time to completion (the total time to connect the sequence of numbers or numbers and letters);

Avg. connection time (the average time needed to connect two neighboring fields when no error was made);

Time(mixed)/Time(digits) (the ratio of the completion time for the second and the first task); and

ATC(mixed)/ATC(digits) (the ratio of the average connection time for the second and the first task).

Executive Maze

The subject is presented with a grid (8×8 matrix) of circles on the computer screen. The object of the task is to find the hidden path through the grid, from the beginning point at the bottom of the grid to the end point at the top. The subject is able to navigate around the grid by pressing arrow keys. The subject is presented with one tone (and a red cross at the bottom of the screen) if they make an incorrect move, and a different tone (and a green tick at the bottom of the screen) if they make a correct move. Each time the subject does the task, the maze is the same. Through trial and error, subjects are required to uncover a hidden pathway linking the start to the end position of the maze. Once subjects reach the end point they are required to repeat the (still hidden) maze from start to finish. Since the task requires coordination of visual, motor and memory skills, it can be used to assess executive function. Subjects continue until they either complete the maze twice in a row with no mistakes, or the test duration of eight minutes runs out (whichever comes first).

Test procedure: The subject is required to discover (by trial and error) a hidden path through a maze and remember it.

Functions measured: Planning, abstraction, foresight, error correction, the ability to choose, try, reject and adapt alternative courses of thought and action; visuo-spatial learning and memory.

Putative brain regions involved: Widespread brain networks.

Practical significance: Ability to plan, strategize and implement complex tasks involving visuo-spatial information.

Scores recorded:

Trials completed—(the number of trials that the subject completed before the task ended or a timeout occurred;

Time to completion (the time the subject took to complete the task twice without error—or until a timeout occurred after 8 minutes);

Path learning time (the time the subject took to discover the hidden path). If no timeout occurred this is the total time excluding the time needed for the last two trials—otherwise it is equal to the total time;

Number of errors (the total number of errors that the subject made); and

Number of overruns (the total number of overrun errors that the subject made). An overrun error occurs if the subject goes in the same direction on a subsequent move but should have changed direction;

Malingering Test (Number Recognition Malingering Test)

This test assesses the capacity to remember words presented on a computer screen. The design of the test ensures that one should be able to get a certain percentage of the trials correct simply by chance. A failure to achieve chance level suggests a deliberate attempt to understate memory capacity. This test requires the subject to recognize words presented on the screen. A score below the level expected by a random choice indicates deception by the subject.

The 1-in-5 Test is designed to detect suboptimal effort or deliberate feigning of impairment. Similar to other symptom validity tests that have an established role in neuropsychological assessment, the 1-in-5 Test requires the patient to select one of a series of numbers that was shown a few seconds earlier. Increased sensitivity is achieved by a chance performance resulting in a score of 80% correct. Scores significantly below 80% can only be achieved by deliberately selecting wrong answers. Low scores provide strong evidence that test results are not valid. The task is simple to perform, even in the context of brain injury. While a high score on the test does not guarantee that other results are valid, as is the case with other similar tests, a good performance increases the likelihood that the patient has provided an optimal performance on tests.

The test interpretation has been divided into three parts based on the results:

1. Scores at or above 90% correct—Testing designed to investigate the validity of responding supported the patient's test performance as a valid indication of current functioning. There was no suggesting of sub-optimal effort or any deliberate attempt to feign impairment.

2. Scores between 68 and 89% correct—On a test designed to investigate validity of responding, there was evidence that the patient did not provide an optimal performance. Given the evidence of inadequate effort, scores on other tests cannot be considered valid indicators of the patient's abilities.

3. Scores below 67% correct—On a test designed to investigate validity of responding, there was strong evidence that the patient was deliberately selecting incorrect responses. Scores on other tests cannot be considered valid indicators of the patient's abilities.

The malingering test measures deliberate underperformance by the subject in order to exaggerate their symptoms.

Further Information Collected

Further information can also be stored in the database 4, including

genetic information taken from swabs or the like could also be stored with the information;

structural MRI (sMRI) data including Dual echo sequence (separation of gray, white matter and CSF), and MPRAGE sequence (volume analysis of individual structures); and

functional MRI (fMRI) including data collected from the Go-Nogo; Auditory oddball; Working memory and Face emotion processing paradigms.

Genetics

Genetics are sampled from the subject's saliva via a cheek swab. Genetic analyses help determine the biological bases of individual differences and mental disease. Genetics of brain function is a field still in its infancy. Variations in the human genome can influence neurotransmitter function and brain structure. With understanding of the genetic bases of mental diseases, prone individuals can benefit from early intervention.

sMRI Protocols

Structural magnetic resonance imaging (sMRI) is used to measure the volumes of gray matter (neurons), white matter (connections) and fluid filled spaces in the brain. It measures the local magnetic fields of water molecules in the brain. The water in different tissue types responds differently to externally applied magnetic fields, enabling measurement of structure at the millimeter scale.

The standard protocol acquires data using 4 different types of MRI contrast that are capable of revealing different aspects of brain cytoarchitecture. These four types of image are:

1) Spin-echo image (dual echo): reflects T2 MRI contrast. Tissue contrast is: csf>grey>white.

2) Proton-density image: reflects the concentration of water. Tissue contrast is: csf>grey>white

3) T1-weighted image: signal intensity is low in tissue with a long T1 and high in tissue with a short T1. Contrast: white>grey>csf.

4) Diffusion Tensor Imaging: gives a variety of contrast that reflects the diffusion speed of water in brain tissue and also the local direction of diffusion in tissues. This latter fact can be used to generate measurements of connectivity (via axons) in the brain.

| | | |
|---|---|---|
| 1) | Dual echo: | |
| | Axial orientation | |
| | 3 mm slice thickness | |
| | No. slices | 60 (no gap) |
| | TR | 7529 ms |
| | TE | 15/105 |
| | Echo train | 7 |
| | Flip Angle | 180 |
| | FOV | 220 mm × 220 mm |
| | Pixel size | 0.87 × 0.86 |
| | NEX | 1 |

Other details: Frequency direction=anterior posterior, acquisition Matrix=252×256, phase encoding L>R, 8/8 rectangular field of view. Acquisition duration: 4 min, 40 sec.

| | | |
|---|---|---|
| 2) | T1 MPrage | |
| | Saggital orientation | |
| | 1 mm slice thickness | |
| | No. slices | 180 (no gap) |
| | Flip angle | 12 |
| | TR | 9.7 ms |
| | TE | 4 |
| | TI | 200 |
| | Matrix | 256 × 256 |
| | FOV | 256 mm × 256 mm |
| | Pixel size | 1.00 × 1.00 |
| | NEX | 1 |

The acquisition duration for the T1 MPrage is about 8 minutes and 20 sec.

3) Repeat the T1 MPrage (exactly as above).

| | | |
|---|---|---|
| 4) | Diffusion Tensor Imaging: | |
| | Axial orientation (same as dual echo) | |
| | 6.5 mm slice thickness | |
| | No. slices | 28 (no gap) |
| | TR | 160 ms |
| | TE | 88 ms |
| | b | 0, 1250 s mm⁻² |
| | d (little delta) | 25 ms |
| | D (big delta) | 31 ms |
| | Matrix | 128 × 128. |
| | FOV | 220 mm × 220 mm |
| | Averages | 4 |

Other details: Fat saturation on, 12 diffusion gradient directions. The acquisition duration of the diffusion tesnsor imaging is about 5 minutes.

This data are saved as DICOM images then transferred electronically to the central database for storage and processing. The above parameters are used to collect the current BRID structural MRI library of 369 images.

fMRI Paradigms

Functional magnetic resonance imaging (fMRI) monitors minute changes in blood flow in the brain that indicate which areas are active during different tasks. It relies on the contrast between the natural magnetic properties of oxygenated versus deoxygenated below to provide a measure of blood oxygen level depended (BOLD) signal change in regions of the brain. Task-related changes in brain activity are measured at a time-scale of about 2-3 seconds and a spatial-scale of one millimeter.

The fMRI paradigms are based on a subset of those used for ERPs. For paradigm 1 (sensory-motor GO-NO GO) the fMRI the stimuli is: GO STIMULI (A)—GREEN PRESS in centre of black screen (TWICE size); NO-GO STIMULI (B)—RED PRESS in centre of black screen (TWICE size); Total of 126 (75%) GO (A) stimuli and 42 (25%) NO-GO (B) stimuli. A and B are grouped into ‘pseudo-blocks’ of 6 stimuli each (ie. to form ‘GO’ and ‘NO-GO’ stimulus blocks)—3 fMRI measurements per block (eg. 1 measurement for 2 stimuli) for a total of 21 GO blocks and 7 NO GO blocks. The task is to tap response box as quickly as possible when GO (GREEN) dot appears and to stop tapping when NO-GO (RED) appears. Blocks are presented in pseudo-random sequence, with constraint that there are no more than two NO-GO blocks in a row (3 fMRI measurements per block). In practice 1 block of each GO and NO-GO. During fMRI, blocks commence after 3 dummy scans. The fMRI parameters are: 84 measurements/volumes in total plus 3 dummy measurements, 15 slices, slice thickness 6 mm (10% gap).

For paradigm 2 (auditory oddball) the fMRI stimuli (for fMRI, presented via Avotec Silent Scan system) is: B Backgrounds: 50 ms 75 dB tone at 500 Hz; T Targets: 50 ms 75 dB tone at 1000 Hz; The task for this paradigm is to count number of stimuli. The sequence is a fixed pseudorandom sequence of B and T with the T preceded by: Low percentage background subaverage (2 p.b.—3 blocks, 3 p.b.—4 blocks, 4 p.b.—3 blocks, Total 10 blocks, 30 backgrounds); High p.b. subaverage (6 p.b.—3 blocks, 7 p.b.—2 blocks, 8 p.b.—2 blocks, 9 p.b.—3 blocks, Total 10 blocks, 75 backgrounds); Stimuli commence after the 3 dummy scans. The total number of stimuli is 125, with 20 T and 105 B (approx. 15%). The fMRI parameters are: 125 measurements/volumes in total plus 3 dummy measurements, 15 slices, slice thickness 6 mm

In paradigm 4A (faces with HAPPY) the fMRI stimuli are: N=Neutral face, any one of 8 persons (Gur stimuli); H=Happy face, any one of 8 persons; S=Startle (tone), duration 50 ms (face stimuli include same 4 females, 4 males). The sequence of the stimulus blocks is:

1. Happy (8 happy stimuli—3 fMRI measurements): 10 repeats, with 5 repeats followed by Happy+tone block,

2. Happy+tone (8 happy stimuli, with startle presented with first stimulus—3 fMRI vols): 5 repeats (this block follows half of the Happy blocks).

3. Neutral (8 neutral stimuli—3 fMRI measurements): 10 repeats

4. Happy+tone (8 happy stimuli, with startle presented with first stimulus—3 fMRI measurements): 5 repeats (this block follows half of the Happy blocks).

TOTAL: 240 stimuli (80 fear, 40 fear+tone, 80 neutral, 40 neutral+tone)=90 fMRI volumes

Blocks are presented in pseudo-random sequence with constraint that fear+tone must always follow a fear block, and neutral+tone blocks must always follow a neutral block. The 8 stimuli are included randomly in each block (each of the 8 faces will appear an equal number of times). Each stimulus is presented for 500 ms (unmasked). The tone is presented for 50 ms coincident with the FIRST FACE STIMULUS in the Happy/Neutral+tone blocks. The fMRI parameters are: 90 measurements/volumes in total plus 3 dummy measurements (93 in total), 15 slices, slice thickness 6 mm (10% gap.

In paradigm 4B (faces with FEAR) the fMRI stimuli are: N=Neutral face, any one of 8 persons (Gur stimuli); F=Fear face, any one of 8 persons; S=Startle (tone), duration 50 ms (face stimuli include same 4 females, 4 males). The sequence of the stimulus blocks is:

1. Fear (8 fear stimuli—3 fMRI volumes): 10 repeats, with 5 repeats followed by Fear+tone block.

2. Fear+tone (8 fear stimuli, with tone presented with first stimulus—3 fMRI measurments): 5 repeats (this block follows half of the Fear blocks).

3. Neutral (8 neutral stimuli—3 fMRI measurements): 10 repeats.

4. Neutral+tone (8 happy stimuli, with tone presented with first stimulus—3 fMRI measurements): 5 repeats (this block follows half of the Neutral blocks).

TOTAL: 240 stimuli (80 fear, 40 fear+tone, 80 neutral, 40 neutral+tone)=90 fMRI measurements

Blocks are presented in pseudo-random sequence with constraint that fear+tonemust always follow a fear block, and neutral+tone blocks must always follow a neutral block. The 8 stimuli are included randomly in each block (each of the 8 persons will appear an equal number of times). Each stimulus is presented for 500 ms (unmasked). The tone is presented for 50 ms coincident with the FIRST FACE STIMULUS in the Fear/Neutral+Startle blocks. The fMRI parameters are: 90 measurements/volumes in total plus 3 dummy measurements (93 in total), 15 slices, slice thickness 6 mm (10% gap)y.

In paradigm 5 (verbal working memory task) the fMRI stimuli is a single capital letter which is one of the four letters B, C, D or G, displayed on a black screen in two different colours (yellow or white). If the same letter occurs twice in a row in yellow, then the second letter is a target. So, the subject must retain in memory the last yellow letter, and when a yellow letter appears, the subject must update their memory (or, if it is a target, press a button). For white letters the subject is not required to do anything (ie. white letters serve as a ‘perceptual’ baseline). The total number of stimuli is 125 with 20 targets. In yellow, there are 21 Bs, 22 Cs, 21 Ds and 21 Gs. In white, there are 10 of each letter. Targets must be separated from each other by at least two letters (because of the fMRI BOLD response). Each letter is a target on 1 in 4.25 occasions. The task is to press button when there is a letter matches the letter ‘one back’. Stimuli commence after the 3 dummy scans. 125 measurements/volumes in total plus 3 dummy measurements, 15 slices, slice thickness 6 mm (10% gap).

See Selected References for Further Detail/Clarifiaction:

1. Williams L M, Kemp A H, Felmingham K, Barton M, Olivieri G, Peduto A S, Gordon E, Bryant R A (in press). Trauma modulates amygdala and medial prefrontal responses to consciously attended fear. Neuroimage.

2. Williams L M, Liddel B J, Kemp A H, Bryant R A, Peduto A S, Meares R A & Gordon E. (in press). An amygdala-prefrontal dissociation of subliminal and supraliminal fear. Human Brain Mapping.

3. Bryant, R A, Felmingham, K L, Kemp, A H, Barton, M, Rennie, C, Gordon E. & Williams, L M (in press). Neural Networks of Information Processing in Posttraumatic Stress Disorder: A Functional MRI Study. Biological Psychiatry.

4. Das P, Kemp A H, Liddell B J, Brown K J, Olivieri G, Peduto A S, Gordon E, Williams L M (In press). Pathways for fear perception: Modulation of amygdala activity by thalamo-cortical systems. Neuroimage.

5. Liddell J, Brown K J, Kemp A H, Barton M J, Das P, Peduto A S, Gordon E and Williams L M (2005). A direct brainstem-amygdala-cortical ‘alarm’ system for subliminal signals of fear. Neuroimage, 24, 235-243.

Reproducibility and Validity Studies

The reproducibility and validity of the above test procedures was tested over two sessions with a total of 21 healthy volunteers (11 males, 10 females, mean age in years=27.76, standard deviation of age=13.47, range=12-57; mean years of education=15.14, standard deviation of education=2.29, range=9-18). The two sessions were conducted four weeks apart. A wide age range was used to address concerns in psychophysiology reproducibility studies that are typically restricted to limited age ranges, without older/younger subjects.

The subjects were screened using standard exclusion criteria, being:
Not having English as primary language.
A personal history of mental illness not related to physical brain injury.
A personal history of physical brain injury.
A personal history of having received a blow to the head that resulted in unconsciousness (within the last 5 years only).
A personal or family history (mother, father, brother, sister, child) of Attention Deficit Hyperactivity Disorder (ADHD), Schizophrenia, Bipolar Disorder or other psychological and/or psychiatric disorder.
A personal history of stroke or neurological disorder such as Parkinson's Disease, Epilepsy, Alzheimer's or Multiple Sclerosis.
A personal history of serious medical conditions related to your Thyroid or Heart, or a history of cancer.
A blood borne illness (HIV, Hepatitis B, Hepatitis C).
A severe impediment to vision, hearing, or hand movement.
A personal history of addiction to drugs such as Heroin, Cocaine or Amphetamines
A personal history of heavy consumption of Marijuana or alcohol.
A personal or family history of genetic disorders.

All subjects completed both psychometric and psychophysiology testing for Session 1 and Session 2. Data acquisition and analysis protocols, and results are reported separately for each testing component.

Reproducibility Summary

Across both EEG and ERP measures, there are no significant changes from Session 1 to Session 2, when key covariates (age, gender) were controlled. Similarly, psychometric measures were also stable across the 4-week repeat period when these key covariates were controlled.

Psychophysiology Acquisition and Analysis

EEG and ERP data were acquired using the standard BRID protocols described previously.

EEG: Both resting eyes closed and eyes open conditions, with parameters of the power spectrum estimated for delta (1.5-3.5 Hz) theta (4-7.5 Hz), alpha (8-13 Hz) and beta (14.5-30 Hz) frequency bands.

ERP: ERPs were included to the auditory oddball and working memory tasks:

Auditory Oddball: target ERPs N100 (80-140 ms), P200 (140-270 ms), N200 (180-320 ms), P300 (270-550 ms)—and background ERPs N100, P200.

Working memory: background ERPs P150 (115-190 ms) and P300 (285-600 ms).

Within-subjects multiple analyses of covariance were conducted with session×condition (e.g. eyes closed/open)×midline, with age and sex as covariates (given robust evidence for relationships between age, sex and psychophysiological function).

Psychophysiology Results

EEG Power: There were no significant differences involving Session for EEG power, when age and sex were controlled for. When age and sex were not included as covariates, the following session effects were observed: Theta power: Session effect (F=16.62, p=0.001); Session by condition interaction of marginal significance (F=4.72, p=0.042).

EEG Frequency: There were no significant effects across the two sessions.

ERP (Oddball): Again, there were no significant effects across the two sessions when age and sex were controlled. When age and gender were not controlled for, the N100 latency for backgrounds was slightly longer in Session 2 by about 5 ms (F=4.92, df=1,18, p=0.04). For targets, both N200 latency (F=4.90, df=1,18, p=0.042) and P300 latency (F=4.84, df=1,17, p=0.042) were slightly longer for Session 2. Together, these data suggest a slight latency shift of the whole waveform in Session 2—a shift that interacts with demographic data.

ERP (Working memory): There were also no significant effects involving Session for P150 and P300 data.

Psychological Data: Procedure and Acquisition

The tests included:

1. Choice Reaction-time

2. Spot the real word test

3. Span of visual memory test

4. Digit span

5. Switching of Attention (parts 1 and 2)

6. Word Interference Test (Stroop)

7. Word Generation (FAS)

These tests generate 16 scores, such that the stringent corrected alpha level is 0.05/16=0.003. Given that scores from the same test might be considered repeated measures, we used an alpha level of 0.05/7=0.007.

The results showed no significant changes across the two sessions for any of the tests. Only when age/gender were not controlled was the following session main effect observed at the corrected alpha level:

Switching of Attention, Part 2 (F=9.47, df=20, p=0.006).

At the uncorrected alpha level, the following session effects were observed:

Spot the Real Word (F=7.18, p=0.014).

Word Generation, FAS (F=5.69, p=0.027).

Memory Recall Total (F=10.27, p=0.004).

Validity of Psychometric Data

The results of the Psychometric testing demonstrate that the database psychological tests (collectively referred to by the trade name IntegNeuro) provide a highly valid method to assess individual differences and changes in cognitive function. There were strong correlations with standard paper-and-pencil measures and the expected differentiation of younger and older individuals.

Validity reflects the degree to which a test actually measures a targeted entity, and it is the ultimate benchmark criterion for any neurocognitive assessment tool. Even in the context of solid reliability a test or battery of tests that fail(s) to measure an intended construct provides no added value.

Two primary methods were followed for establishing validity of the database:

Testing the expected correlations with a previously developed (‘traditional’) version of the test.

Identification of performance differences on the test that exist across one or more ‘known’ group (e.g. it has been established that older individuals perform more poorly than younger individuals on cognitive tests that involve mental speed and flexibility).

A total of 50 healthy adults completed both:

1) The preferred embodiment psychological tests (forming IntegNeuro)

2) Previously developed cognitive measures typically administered in research and clinical settings, including paper-and-pencil tests described in detail in primary textbooks in the field of Neuropsychology and Neurology (e.g. Muriel D. Lezak Neuropsychological Assessment Fourth Edition, Oxford University Press, etc). These were selected according to the following two criteria: a) the tests measured the same cognitive construct as the tests of IntegNeuro; and b) the tests were among the most common cognitive measures (Lezak).

Tests and Procedure

The IntegNeuro tests include finger tapping, word generation (verbal fluency), spot the word test, memory recall, digit span test and switching of attention. In one half of the cases (25 individuals), the IntegNeuro battery was administered at the first visit, and four weeks later the previously developed paper-and-pencil measures were administered at a second visit. The other half of the cases (25 individuals), the paper-and-pencil measures were administered first and IntegNeuro was administered second. The order of administration (IntegNeuro vs. paper/pencil) was determined by random assignment to avoid any potential bias.

Validity was assessed by examining the degree of similarity in performance on both test types. Correlational analyses were computed for the entire group (50) and separately for individuals under the age of 46 (range=22-45) and individuals 46 and older (range=46-80). The purpose of the separate analyses for age was to determine with certainty that the validity of the IntegNeuro measures was not influenced by older age. Validity was also assessed by examining differences in performances on the individual tests between young individuals and older individuals.

Results

Each IntegNeuro test was correlated significantly with the relevant paper-and-pencil measure. In each case, there was a statistically significant degree of overlap between the two approaches. In several cases, the degree of overlap was substantial (correlation greater than 0.75). Importantly, the strength of the correlations was dot affected by age of the participants. All significant correlations remained when the two groups were examined separately. The validity of IntegNeuro was also supported by the results of the between-group differences. For each IntegNeuro and equivalent paper-and-pencil measure, the younger individuals performed statistically better than older individuals.

Summary

Upon collation, the preferred embodiment provides for a commercially valuable standardised database that provides relevant information that is evidence based. The database can then be utilized as an analysis basis. The provision of a large number of tests allows for the covariances between tests to be investigated and exploited.

The data acquired can be standardised and subject to quality control processes so as to ensure its uniformity across sites. Preferably, identical acquisition protocols and identical equipment is utilised at each site and centrally processed by the one main server 4. The server can include all analysis tools—simulation models and mathematical tools for averaging and sub-averaging data and assessing statistical outputs.

By centralizing the storage and analysis, a diverse range of analysis can be carried out. This includes insights into disorders, insight into the effects of existing and new drugs on the brain and application as a screening device for many aspects of cognition.

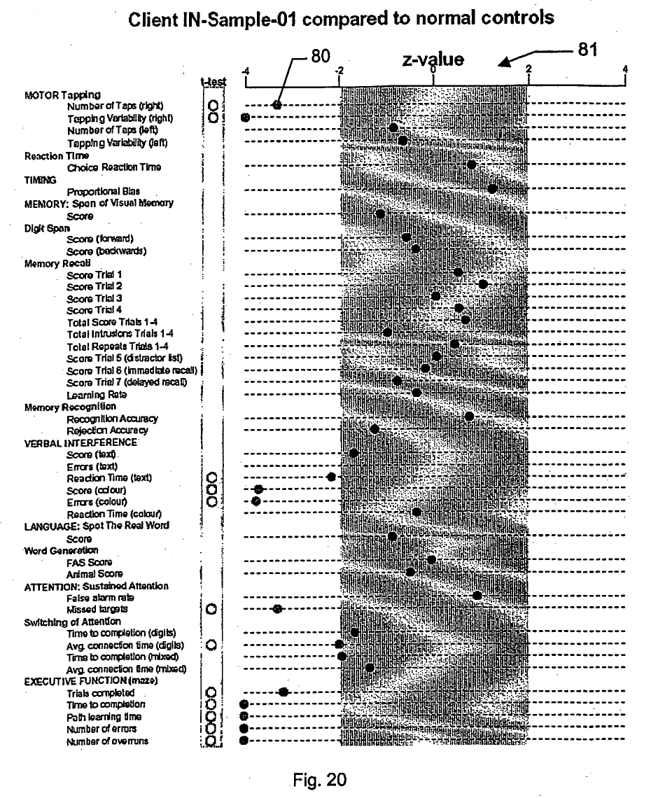

For an individual patient under test, the corresponding test data can than be acquired and analysed by the server engine and compared to the database and a report generated the highlighting areas of deviance. An example of such a report is illustrated in FIG. 20 where a patient's results are indicated 80 in comparison to an average range 81 for a series of conducted experiments. By viewing such reports it is possible to view areas of concern in light of the indicators outlined below:

Depression

Depression has been characterized by several structural and functional brain abnormalities. Structural MRI studies of patients with depression have shown increased white matter, CSF and temporal volume, as well as an increased Sylvian fissure. Decreased total brain and relative prefrontal lobe volume have also been found, as well as hyperintensities in the periventricular pons and frontal brain region, the putamen and globus pallidus. In addition, functional MRI studies have found reduced activation of the left prefrontal region.

Abnormalities of electrical brain show decreased EEG delta and increased theta activity, while both increased and decreased alpha and beta activity have been reported, with differences in beta activity in some studies reported only over frontal regions. Abnormalities of functional brain asymmetry include greater right than left frontal activation, greater variability over the right than left hemisphere, and greater left than right alpha, theta and beta values. Event-related potentials (ERPs) show decreased amplitudes of the ERP N1, P2, N2 and P3 components. However, results are contradictory, with several studies finding no difference in the amplitudes of any ERP components. In addition, the latency of the P1 component has been reported to be increased, and the P2 and P3 components to be decreased. Asymmetries of ERP components have also been reported, with the amplitude of the N2 component being greater over the right than the left hemisphere, and P3 latency being greater over the left than the right frontal region. Numerous studies have found delayed reaction times in patients with depression.

Patients with depression have also been repeatedly found to show abnormalities in arousal levels, showing decreased baseline levels of skin conductance but increased heart rate. Depression has also been characterized by deficits on several neuropsychological measures, including psychomotor speeds, verbal fluency, episodic memory, working memory short-term memory, sustained attention, divided attention, selective attention, response inhibition and executive function.

Early theories stated that depression was associated with depletion of brain neurochemicals such as norepinephrine and serotonin. Depletion of these chemicals is relevant to the action and maintenance of antidepressant responsiveness. However, reduction of monoamine levels alone cannot account for the etiology of depression. For example, depletion of monoamines in most healthy individuals does not induce the condition. Alternatively, there is evidence to suggest that other neurotransmitters or regulatory systems and their signal transduction pathways contribute to the illness, in particular stress. Stress, hippocampal function and depression may be intertwined [Miller H L, Delgado P L, Salomon R M, Berman R, Krystal J H, Heninger G R, Charney D S (1996) Clinical and biochemical effects of catecholamine depletion on antidepressant-induced remission of depression, Arch Gen Psychiatry, 1996February;53(2):117-28; Flugge G, Van Kampen M, Mijnster M J, Perturbations in brain monoamine systems during stress, Cell Tissue Res. 2004January;315(1):1-14; Mizoguchi K, Ishige A, Aburada M, Tabira T, Chronic stress attenuates glucocorticoid negative feedback: involvement of the prefrontal cortex and hippocampus, Neuroscience 2003;1 19(3):887-97].

Bipolar Disorder

Structural MRI studies have found patients with bipolar disorder to show increased abnormal white matter, white matter hyperintensities, ventricles, temporal and frontal sulci and an increased Sylvian fissure, as well as decreased intracranial and pituitary volume. In addition, functional MRI studies have found increased left amygdala activation to masked faces, normalising with treatment, and decreased prefrontal activation during depressive periods.

Abnormalities in brain function, as indexed by electrical brain activity, have also been found. The ongoing EEG of bipolar disorder patients has been found to show increased delta, theta and beta activity and decreased alpha activity. In addition, opposite slow-wave frontotemporal asymmetries have been reported between depressive and manic states. Studies of event-related potentials (ERP) have found the amplitude of the P3 component to be decreased anteriorly, and P3 latency has been reported to be increased. Asymmetry in ERP components has also been reported, with N1 amplitude being greater to stimuli presented to the left than the right hemisphere. Reaction times to stimuli have also been reported to be increased.

Arousal, as indexed by skin conductance level, has also been reported to be decreased in bipolar disorder, both at baseline levels and in reaction to stimuli. Bipolar disorder has also been associated with deficits on several neuropsychological measures, including motor coordination, fine motor skills, verbal fluency, verbal learning, verbal memory, sustained attention, decision making and executive function.

Bipolar disorder is associated with alterations in central nervous system (CNS) function from the level of large-scale brain circuits to intracellular signal transduction mechanisms within individual cells. Signal transduction pathways, which are important mediators of neurotransmitter generated signals. Regulation of signal transduction within critical regions of the brain by lithium affects the function of multiple neurotransmitter systems and may thus explain lithium's efficacy in protecting susceptible individuals from spontaneous, stress-induced, and drug-induced cyclic affective episodes [Berns G S, Nemeroff C B, The neurobiology of bipolar disorder, Am J Med Genet, Nov. 15, 2003;123C(1):76-84; Manji H K, Potter W Z, Lenox R H. Signal transduction pathways, Molecular targets for lithium's actions, Arch Gen Psychiatry, 1995July;52(7):531-43; Lachman H M, Papolos D F, Abnormal signal transduction: a hypothetical model for bipolar affective disorder, Life Sci. 1989;45(16):1413-26].

Schizophrenia

Schizophrenia has been characterized by a diversity of structural and functional brain abnormalities. Studies of brain structure have found increased ventricular volume and increased frontal and temporal sulcal size, as well as a decreased volume of the hippocampus, amygdala and gray matter of the temporal lobe and sub-cortical frontal and parietal regions. Functional fMRI imaging studies have also reported decreased amygdala and medial prefrontal cortex activity, and dorsolateral prefrontal cortical activity has been found to be dysfunctional during working memory tasks.

Abnormalities of electrical brain activity are characterized by increased EEG delta and theta activity and decreased alpha activity. Target event-related potentials (ERP) show increased amplitude of the P2 component and decreased amplitude of the N1, N2 and P3 components, with the amplitude of the P3 component being reported to be larger over the temporal region of the right than the left hemisphere, and more decreased posteriorly. In addition, the latencies of the P2, N2 and P3 components have been found to be increased. In response to background tones, people with schizophrenia have been found to show decreased N1 amplitude as well as decreased P2 and N2 latency. Gamma phase synchrony has also been found to be altered in schizophrenia, with decreased amplitude of the G1 component over the right hemisphere, and decreased amplitude of the G2 component over the frontal region of the left hemisphere. Delayed and diminished ERPs in response to faces, in particular to negative affect, have also been found. Delayed reaction times to stimuli have also been reported, and have been related to both symptom severity and diagnostic outcome.

Schizophrenia has also been associated with altered levels of arousal, as indexed by measures of skin conductance level and heart rate. People with schizophrenia have been found to show decreased and delayed skin conductance responses to auditory stimuli as well as an increased proportion of non-responders, however they have also been found to show increased responses to faces displaying negative affect. They have also shown decreased habituation to startle stimuli and decreased prepulse inhibition, increased baseline heart rate levels, decreased heart rate responses to stimuli and increased heart rate variability.

Schizophrenia has also been characterized by deficits on several neuropsychological measures, including verbal fluency, verbal memory, working memory short-term memory, and motor skills, trail making as well as an inhibitory disturbance reflected in the Stroop.

Pharmacological probes have identified dopamine receptor stimulants and glutamate receptor inhibitors as models for Schizophrenia. Interestingly, genetic studies point more towards a role for the glutamate pathway rather than the dopamine pathway in schizophrenia [References: Tunbridge E, Burnet P W, Sodhi M S, Harrison P J; Catechol-o-methyltransferase (COMT) and proline dehydrogenase (PRODH) mRNAs in the dorsolateral prefrontal cortex in schizophrenia, bipolar disorder, and major depression; Synapse 2004February;51(2):112-8].

Negative symptoms have been associated with decreased prefrontal D1 receptors and with NMDA inhibition. Addition of serotonin subtype receptor inhibitors and NMDA stimulants in combination with D2 receptor blockade improve negative symptoms. The locations of NMDA and 5HT receptors that mediate these actions have not been identified [Arnt J., Skarsfeldt T: Do novel antipsychotics have similar pharmacological characteristics? A review of the evidence. Neuropsychopharmacology 18: 63-101,1998].

Schizophrenia has been conceptualized as a failure of cognitive integration, and abnormalities in neural circuitry (particularly inhibitory interneurons) have been proposed as a basis for this disorder [Benes F M, Berretta S: GABAergic interneurons: implications for understanding schizophrenia and bipolar disorder; Neuropsychopharmacology 2001July;25(1):1-27].

A single molecule in the brain may be responsible for multiple neurotransmitter changes in Schizophrenia the molecule identified, DARPP-32 [Svenningsson, P., et al, Diverse psychotomimetics act through a common signalling pathway, Science, 302, 1412-1415, (2003)].

Anxiety Disorders

The most consistent findings in Anxiety Disorders have been associated with autonomic nervous system abnormalities. Nevertheless, structural MRI studies have found patients with anxiety disorders to show decreased temporal lobe volume. Abnormalities of electrical brain activity have been found. Increased EEG delta, theta and alpha activity and decreased beta activity. Anxiety disorders have also been associated with greater right than left hemisphere activity, with reduced anxiety being associated with increased activity over the left frontal region. In addition, panic disorder has been associated with abnormalities of the non-dominant temporal lobe. Event-related potentials (ERP) have been contradictory. The amplitude of the N1 component has been found to be increased, however several studies have found the amplitudes the N2 and P3 components to be both increased and decreased. Differences between anxiety disorders have also been found, with panic disorder showing a frontal increase in P3 amplitude, and also showing greater ERP latencies than in generalized anxiety disorder. Anxiety disorder patients have also been repeatedly found to show delayed reaction times to threat-related stimuli, with one study finding such a delay only to stimuli presented to the left hemisphere.

Anxiety disorder patients have also been found to show increased arousal, as indexed by measures of skin conductance and heart rate. Both increased baseline arousal levels and increased reactions to threat-related stimuli have been found, as well as an increased rate of spontaneous skin conductance fluctuation. In addition, differences between anxiety disorders have been found, with phobia patients showing faster habituation to stimuli and generalized anxiety and panic disorder patients showing slower habituation. Differences between anxiety disorders in heart rate variability have also been found, with anxiety disorder patients generally showing decreased variability and panic disorder patients showing increased variability.

Anxiety disorders have also been characterized by deficits on several neuropsychological measures, including visual memory and divided and selective attention. Anxiety disorder patients have also been found to show a bias towards emotional and threat-related stimuli, as indexed by specialized Stroop tasks.

There is some evidence that the neurobiologic basis of generalized anxiety disorder may involve abnormalities in neurochemical, neuroendocrine, neurophysiologic, and neuroanatomic factors. Maladaptive responses to stressful stimuli have been observed in the locus-ceruleus-norepinephrine-sympathetic nervous system, the hypothalamic-pituitary-adrenocortical axis, and the cholecystotin system. Abnormalities in other important CNS modulators, such as 5-HT and gamma-aminobutyric acid, may also be involved in the biology of generalized anxiety disorder [Hidalgo R B, Davidson J R, Generalized anxiety disorder, An important clinical concern, Med Clin North Am. May 2001, 85(3), 691-710; Brawman-Mintzer O, Lydiard R B, Biological basis of generalized anxiety disorder, J Clin. Psychiatry, 1997, 58 Suppl 3, discussion 26 p:16-25].

Post-Traumatic Stress Disorder (PTSD)

The main structural abnormality found in MRI studies has been a decreased volume of the hippocampus. In addition, functional MRI studies have found increased amygdala activity,decreased hippocampal and medial prefrontal cortical activity and when presented with trauma-related stimuli, increased activity in the visual cortices. Several ERP abnormalities have been found. Latencies of the N2 and P3 components have been found to be increased. P2 and P3 amplitudes have been found to be decreased and N2 amplitude to be increased. Trauma-related stimuli reveal a different pattern, with N1 and P3 amplitudes being increased. A slowed reaction time to stimuli in the auditory oddball paradigm has also been found in PTSD. PTSD patients have additionally been found to show increased levels of arousal, as indexed by increased baseline heart rate and skin conductance levels and increased skin conductance and heart rate responses to auditory startle stimuli and trauma-related stimuli. PTSD has been associated with lower IQ levels, and PTSD patients have been found to show both long- and short-term memory deficits, particularly working memory.

Posttraumatic stress disorder is a disorder with an identifiable etiological factor (exposure to a traumatic event) and with a complex symptomatology (i.e. intrusive memories, avoidance, hyperarousal) that suggests dysfunction in multiple psychobiological systems.

Acute and chronic stress showing that traumatic experiences can produce long-lasting alterations in multiple neurochemical systems. Neurotranmitters systems that seem to be involved include serotonin, noradrenaline and dopamine [Grillon C, Southwick S M, Charney D S, The psychobiological basis of posttraumatic stress disorder, Mol. Psychiatry, September 1996, 1(4) p.278-97].

Neuroimaging studies in PTSD with the most replicated findings showing decreased medial prefrontal cortical function in PTSD. Other replicated findings include decreased inferior frontal gyrus function, decreased hippocampal function, increased posterior cingulate function, and, in some behavioral paradigms, increased amygdala function. Several studies have now shown changes in structure (smaller volume) of the hippocampus in PTSD [Bremner J D Neuroimaging studies in post-traumatic stress disorder, Curr Psychiatry Rep. August 2002, 4(4) p.254-63].

An amygdala-locus coeruleus-anterior cingulate circuit may be consistent with evidence for chronic noradrenergic activation documented in PTSD patients [Hamner M B, Lorberbaum J P, George M S, Potential role of the anterior cingulate cortex in PTSD: review and hypothesis Depress Anxiety, 1999 9(1) p.1-14]

Attention Deficit Disorder (ADHD)

Patients diagnosed with ADHD have been found to show decreased total brain volume as well as decreased volume of specific brain regions, including the cerebellum and anterior corpus callosum. Abnormalities of fMRI brain function show methylphenidate to selectively increase caudate and putamen activity during performance on go/no-go tasks. Increased EEG theta activity over frontal and central brain regions, as well as increased alpha and decreased beta activity. Normal age-related changes in the ongoing EEG have also been found to be delayed. Event-related potentials (ERP) abnormalities include decreased amplitudes of the N1, P2, N2 and P3 components, with a decreased P3 amplitude being most commonly reported and some studies finding an increased anterior P3 amplitude. The latency of the G1 gamma phase synchrony component has also been found to be decreased and more pronounced posteriorly, and its amplitude to be increased.

Reaction times of ADHD patients have been found to be longer and more variable than those of controls. ADHD patients also show decreased arousal, indexed by decreased baseline skin conductance levels and decreased task-related and non-specific skin conductance responses.

ADHD patients have also been found to show a different neuropsychological profile to controls, characterized by deficits on tasks involving verbal learning, verbal fluency, short-term memory and disturbed inhibition reflected in Stroop tasks.

The effectiveness of stimulant drugs, along with animal models of hyperactivity, point to catecholamine neurotransmitter disruption as at least one source of ADHD brain dysfunction. Although not entirely sufficient, changes in dopaminergic and noradrenergic function appear necessary for the clinical efficacy of pharmacological stimulant treatments (such as methylphenidate and dextroamphetamine) for ADHD, providing support for the hypothesis that alteration of monoaminergic transmission in critical brain regions may be the basis for therapeutic action in ADHD [Swanson J M, Role of executive function in ADHD, J Clin Psychiatry December 2003, 64 Suppl 14, p.35-9; Biederman J, Faraone S V, Current concepts on the neurobiology of Attention-Deficit/Hyperactivity Disorder, J Atten Disord 2002, 6 Suppl 1 p. S7-16; Molecular genetic data and imaging studies suggesting that the dopamine receptor (DRD4) gene, dopamine transporter/gene (DAT1) and alpha-2A adrenergic receptor genes may be relevant for ADHD; Roman T, Schmitz M, Polanczyk G V, Eizirik M, Rohde L A, Hutz M H, Is the alpha-2A adrenergic receptor gene (ADRA2A) associated with attention-deficit/hyperactivity disorder? Am J Med Genet, Jul. 1, 2003, 120B(1) p. 116-20; Krause K H, Dresel S H, Krause J, la Fougere C, Ackenheil M, The dopamine transporter and neuroimaging in attention deficit hyperactivity disorder, Neurosci Biobehav Rev. Nov. 27, 2003 (7), p. 605-13]

Autism

Autism has been characterized by several structural and functional brain abnormalities. Studies of brain structure have found Autistic individuals to show increased total brain volume and increased volume of the lateral and third ventricles, as well as decreased volume of the midbrain, medulla oblongata, corpus callosum, amygdala, caudate nuclei and left planum temporale. There have also been contradictory reports, with some studies reporting an increased volume of the cerebellum and cerebullar hemispheres, and others reporting a decreased volume of the cerebella.

Differences in brain function have also been found. Autistic individuals have frequently been reported to show decreased amygdala activity in response to facial emotion, with less frequent reports of additional decreased activity in the inferior occipital and superior temporal gyri and the left cerebellum. Autistic individuals have also been found to not show normal fusiform gyrus activity during facial discrimination. Differences in brain function in autism have also been found in studies of electrical brain activity. The ongoing EEG of autistic individuals has been found to show less theta and alpha activity over frontal and temporal regions, with the reduction in theta activity being more prominent over the left hemisphere. Autistic subjects also show reduced or reversed hemispheric activity, particularly during cognitive tasks, and do not show normal left hemisphere specialization for verbal tasks. Event-related potentials (ERPs), changes in the ongoing EEG in response to external stimuli, have also been found to be altered in autism. The amplitudes of the P3 and P3b ERP components have been found to be decreased, and the latency of the N1 component to verbal stimuli over the left temporal region has been found to be increased. Autistic subjects have also been found to show differences in reaction times to stimuli, showing delayed and more variable reactions in serial reaction time tasks, faster anticipatory reaction times and faster reaction times to more complex tasks, resulting from a failure to adjust to changing task difficulty.

Autistic individuals also show decreased arousal, as indexed by measures of heart rate and skin conductance. They show decreased skin conductance responses to both novel and threatening stimuli, as well as decreased habituation and an increased proportion of non-responders. Autistic individuals have been found to show a unique pattern of response, responding with large amplitudes and fast recovery They have also shown differences in heart rate, with a decreased deceleration to auditory stimuli and greater variability. Autism has also been characterized by deficits on several neuropsychological measures, including verbal fluency, attention shifting, executive functions (especially planning) and motor skills, as well as excessive response inhibition.

Relatively few studies have investigated brain structure and function in borderline personality disorder, however several abnormalities have been found. A structural MRI study found decreased volume of the hippocampus and the amygdala, and an fMRI study, evaluating response to negative emotion, found increased amygdala activation and additional activation of the medial and inferolateral prefrontal cortex, not seen in controls. The latency of the P3 ERP (event-related potential) component has been found in several studies to be increased. Arousal, as indexed by skin conductance levels, as been found to be decreased in reaction to both auditory startle and emotional stimuli in borderline personality disorder. No difference in reaction times to stimuli or performance on neuropsychological tasks has been found.

Alzheimer's Disease

Alzheimer's Disease is a form of dementia that accounts for more than 50% of all cases of dementia. It strikes individuals of all socioeconomic backgrounds and spares no major cultural subgroup. Individuals who reach age 65 have a lifetime risk of 5-10%. Onset may be heralded by impaired performance in intellectual demanding tasks at work or a change in personality, reflecting a response to these early deficits. Mild depression occurs in the early stages of the disease process in 30-50% of cases.

Alzheimer's disease has been characterized primarily by structural, but also functional and psychometric patterns of disturbance. Structural MRI studies have found an increased volume of the lateral ventricles and ventricular and sulcal cerebrospinal fluid (CSF), as well as a decreased volume of numerous brain structures (including the hippocampus), amygdala, limbic structures, temporal lobe, left frontal lobe, corpus callosum) and decreased cortical gray matter. The degree of brain atrophy in Alzheimer's disease has been related to symptom severity, and the extent of increased sulcal CSF associated with neuropsychological test scores. Studies of fMRI functional have found enhanced activation of the left dorsolateral prefrontal cortex and bilateral cingulate during phonological tasks, and a lack of temporal and prefrontal lobe activity to visual stimuli (compared to controls). Dysfunction of hippocampal activity has also been reported.

Abnormalities of electrical brain activity have shown increased EEG delta and theta activity as well as decreased alpha and beta activity. The differences in beta and theta activity have, in some studies, only been found over temporal and temporo-occipital regions, while increased delta amplitude has been reported to be more prominent anteriorly and superiorly. Alpha and beta sources have also been reported to show a shift towards more anterior and superior regions. Alzheimer's disease has also been characterized by reduced interhemispheric coherence across all frequency bands, reduced intrahemispheric coherence in the delta and theta bands and reduced temporo-occipital coherence. Event-related potentials (ERP) changes include decreased P3 ERP amplitude, increased latency and an increased rate of latency decline due to normal aging. Alzheimer's disease patients have also been repeatedly found to show delayed reaction times to stimuli.

Alzheimer's disease has been characterized by deficits in many neuropsychological measures, including verbal fluency, verbal memory, episodic memory, short- and long-term memory, divided attention, selective attention, attention-switching, motor skills, response inhibition and executive function.

Although the cerebral cortex is the primary target in AD, degeneration of subcortical (deep brain) structures may also contribute. There have been noted decreases in many neurotransmitter systems in Alzheimer's disease, although these changes are almost certainly due to a secondary to loss of relay systems (projection neurons) from specific sub-cortical structures. Specific neurotransmitter system changes in AD include[Young A, Penny J J: Neurotransmitter receptors in Alzheimer disease, in Alzheimer Disease. Edited by Terry R D, Katzman R, Bick K L, New York, Raven, 1994, p.293-303]:

A loss of neurotransmitter projections to the amygdala. The amygdala is involved in motivation and emotional behavior.

Extensive cell loss in the noradrenergic locus coeruleus, which richly innervates the cortex, has been associated with depressive symptoms.

Changes in the serotonergic raphe nuclei, this may explain impairments in circadian and sleep rhythms.

Unpredictable changes in the cholinergic outputs from the nucleus basalis of Meynert. The nucleus basalis of Meynert provides the major cholinergic input to the cortex and is important for memory.

Closed Head Injury

Patients suffering closed head injury (CHI) have been reported to show several structural and functional brain abnormalities. The most common structural abnormality found has been diffuse axonal injury. The ongoing EEG of CHI patients has been found to show increased theta activity, and the amount of delta activity has been found to be positively correlated with white matter injury. Changes in event-related potentials (ERP) have also been reported in CHI, with the P3 component showing reduced amplitude and increased latency. Delayed reaction times to stimuli have also been repeatedly found in CHI. CHI has been characterized by deficits on several neuropsychological measures, including short- and long-term memory, executive function, fine motor skills and complex language skills, as well as reduced IQ and delayed processing for multiple tasks.

Epileptic Syndromes

Although epileptic syndromes differ pathophysiologically, common ictogenesis-related characteristics consist of increased neuronal excitability and synchronicity. Alterations of synaptic functions and intrinsic properties of neurons are common mechanisms underlying hyperexcitability in the brain. An imbalance between glutamate and gamma-aminobutyric acid neurotransmitter systems can lead to hyperexcitability. Catecholaminergic neurotransmitter systems and opioid peptides also play a role in epileptogenesis [Engelborghs S, D'Hooge R, De Deyn P P, Pathophysiology of epilepsy, Acta Neurol Belg, December 2000, 100(4) p.201-13].

Parkinson Disease

Functional connectivity between basal ganglia and cerebral cortex in humans is dependent on dopamine. An increase in dopamine in these subcortical areas result in tremor and decreases in dopamine result in rigidity. Specifically, movement is dependent on movement-related frequency-specific changes in synchronization occur in the basal ganglia and extend to involve subcortico-cortical motor loops that are dependent on dopamine. In Parkinson's disease a depletion of dopamine interferes with this synchronization between cortical and subcortical motor areas. Local, dopaminergic neuronal groups in the retina, basal ganglia and frontal cortical memory system are affected in Parkinson's disease and may underlie cognitive impairment, visual disturbances, depression and anxiety [Cassidy M, Mazzone P, Oliviero A, Insola A, Tonali P, Di Lazzaro V, Brown P, Movement-related changes in synchronization in the human basal ganglia, Brain, June 2002;125(Pt 6):1235-46; Bodis-Wollner I, Neuropsychological and perceptual defects in Parkinson's disease, Parkinsonism Relat Disord, August 2003, 9 Suppl 2 p. S83-9].

The EEG, ERP and fMRI measures and other measures of brain body function can be part of the standardised methodology with the scoring of data being based on established method for multi variate data analyses. Further specifics of the tests and methods used can be modified as new methods of analysis become available.

### CONCLUSION

The foregoing describes only one embodiment of the present invention, modifications obvious to those skilled in the art can be made thereto without departing from the scope of the invention. It will be readily evident that other forms of tests could be provided and the tests set out may not necessarily themselves be utilized.

The methods and apparatus described herein, and/or shown in the drawings, are presented by way of example only and are not limiting as to the scope of the invention. Unless otherwise specifically stated, individual aspects and components of the calibration methods may be modified, or may have been substituted therefore known equivalents, or as yet unknown substitutes such as may be developed in the future or such as may be found to be acceptable substitutes in the future. The calibration methods may also be modified for a variety of applications while remaining within the scope and spirit of the claimed invention, since the range of potential applications is great, and since it is intended that the present calibration methods be adaptable to many such variations.

It will be appreciated that the illustrated procedures for brain analysis and functional disorder identification described above at least substantially provides a method of obtaining and collating data to be used as a comparative tool on a global scale for brain-related disease and dysfunction.

1. A method of providing diagnosis capability, diagnosis of the effects of treatment or diagnosis of distinctive capabilities of a test subject, the method comprises the steps of:
(a) carrying out a series of tests on a group of subjects of at least two modal measures, said modal measures comprising brain-body function, brain structure, neuropsychological, personality, genetics, personal history, performance and behaviour; and
(b) examining the inter-relationships between said modal measures to output an analysis of the inter-relationships of two or more measures of the tests results of said group of subjects.

2. A method as claimed in claim 1 further comprising the steps of:
(c) examining a test subject on at least two of said modal measures;
(d) analysing the results of step (c) relative to the test results of said group of subjects to determine a distinctive pattern of results for said test subject.

3. A method as claimed in claim 2 wherein said test subject is examined on the same series of tests as carried out on said group of subjects.

4. A method as claimed in claim 2 wherein said test subject is examined on a subset of the series of tests as carried out on said group of subjects.

5. A method as claimed in claim 1 wherein said test subjects are geographically dispersed.

6. A method as claimed in claim 1 wherein the number of control subjects is at least 100.

7. A method as claimed in claim 1 wherein said step (a) further includes the measuring of electromagnetic signals emanating from the subject's brain in response to various interactive tasks carried out by the test subject.

8. A method as claimed in claim 7 wherein said external stimuli include a series of interactive tests conducted by the subject.

9. A method as claimed in claim 7 wherein the measured electromagnetic signals are subjected to signal processing to extract measurements of at least one of delta, theta, alpha, beta gamma frequency ranges for comparison with corresponding ranges of said test subjects.

10. A method as claimed in claim 9 further comprising detecting abnormal power levels in said frequency ranges.

11. A method as claimed in claim 7 further comprising extracting event related potentials from said electromagnetic signals.

12. A method as claimed in claim 7 wherein said interactive tests include at least one of:
a Resting EEG test; a habituation paradigm test, an efficiency of target processing test, a visual tracking task, an inhibition test, a conscious and subconscious processing of facial emotions test, a memory and sustained attention test, a planning and error correction test an a fight and flight reflex test.

13. A method as claimed in claim 7 further comprising the step of conducting a gamma phase synchrony analysis of said electromagnetic signals.

14. A method as claimed in claim 7 further comprising the step of extracting tonic or phasic effects from said electromagnetic signal.

15. A method as claimed in claim 7 wherein said series of tests include a series of information processing tasks, with information designed to be processed over varying periods of time.

16. A method as claimed in claim 7 wherein said electrical signals are measured at multiple locations on the head of a patient and combined together.

17. A method as claimed in claim 7 further comprising recording genetic, structural MRI and functional MRI information for said test patient.
